(19) United States
(12) Patent Application Publication
(10) Pub. No.: US 2004/0204394 A1
(43) Pub. Date: Oct. 14, 2004

(54) Triptolide analogues

(75) Inventor: Gevork Minaskanian, Richmond, VA (US)

Correspondence Address: STERNE, KESSLER, GOLDSTEIN & FOX PLLC, 1100 NEW YORK AVENUE, N.W., WASHINGTON, DC, 20005, US

(73) Assignee: Aderis Pharmaceuticals, Inc.

(21) Appl. No.: 10/411,365

(22) Filed: Apr. 11, 2003

(51) Int. Cl. A61K 31/395; A61K 31/38; A61K 31/336

(52) U.S. Cl. 514/183; 514/475; 548/958; 549/90; 549/332

(57) ABSTRACT

Triptolide analogues are described, including compounds of Formula I:

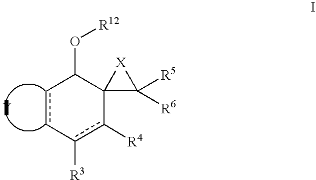

or a solvate, hydrate or pharmaceutically acceptable salt thereof; wherein R³-R⁶, X and Y are set forth in the specification. The compounds of the invention are potent 15-lipoxygenase inducers. Pharmaceutical compositions of the compounds are also described. Also provided are methods for the treatment or prophylaxis of inflammatory and autoimmune disorders in a mammal by administering compounds of the invention.

### BACKGROUND OF THE INVENTION

1. Field of the Invention

The present invention relates to novel triptolide analogues useful in the treatment of inflammatory and autoimmune disorders.

2. Background Art

Various extracts from the Chinese herb Trypterygium wilfordii have been shown to possess immunsuppressive and antiinflammatory properties. In particular, the compound triptolide has potent antileukemic and immunosupressive activities (Kupchan et al., J. Am. chem. Soc. 94:7194 (1972) and Yang et al., Int. J. Immunopharmacology 14(6):963 (1992)). Triptolide contains an unusual triepoxide moiety and an α, α-unsaturated γ-lactose in a diterpene skeleton (Yang et al., Tetrahedron Letts. 38(39):6865 (1997)). In particular, the triepoxide portion of triptolide has been shown to be important for its immunosuppressive activity (Yu et al., Acta Pharm. Sinca 27(11): 830 (1992)). Members of the triptolide family of compounds can induce the production of 15-lipoxygenase (15-LO) in A549 cells. Formation of 15-LO stimulates the formation of 15-S-HETE, which is known to be a natural antiinflammatory agent.

The triptolide compounds are structurally complex molecules and are difficult to purify in useful quantities from its natural source. Moreover, these compounds are difficult to synthesize in practical yields. It would therefore be desirable to find triptolide analogues that simpler in structure and relatively easy to synthesize. In addition, it is desirable to synthesize novel triptolide analogues which have potent antiinflammatory activity and are potent immunosuppressants.

### BRIEF SUMMARY OF THE INVENTION

The present invention is directed to novel triptolide analogue compounds having Formulae I-XXIV (below) and their pharmaceutical compositions. Also provided are processes for preparing the compounds of Formulae I-XXIV. The novel compounds of the present invention havepotent antiinflammatory activity and are potent immunosuppressants.

Also provided are methods for the treatment or prophylaxis of an inflammatory disorder or an autoimmune disorder in a mammal, comprising administering to the mammal an effective amount of a compound of the invention.

### DETAILED DESCRIPTION OF THE INVENTION

Compounds of the present invention include triptolide analogue compounds of Formulae I-XXIII:

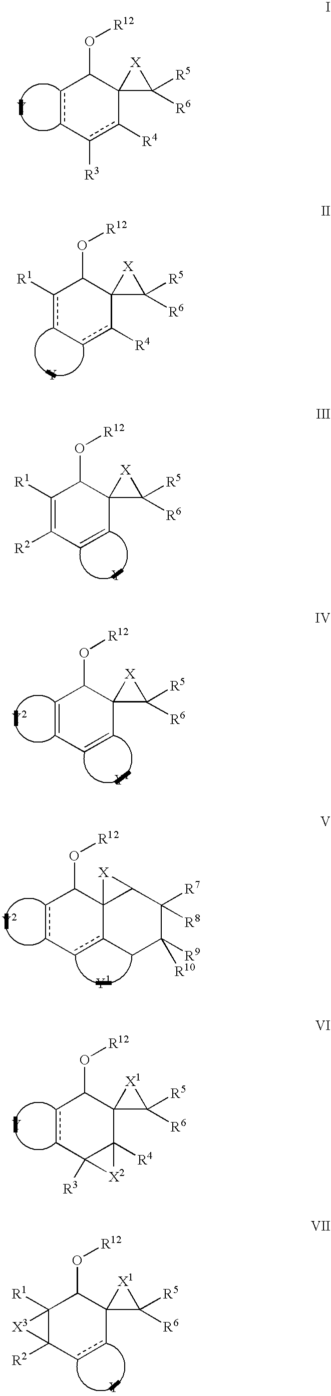

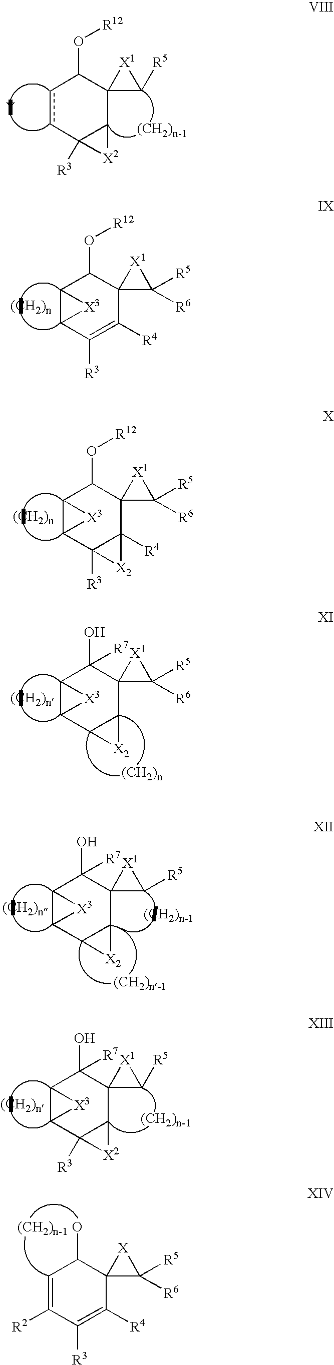

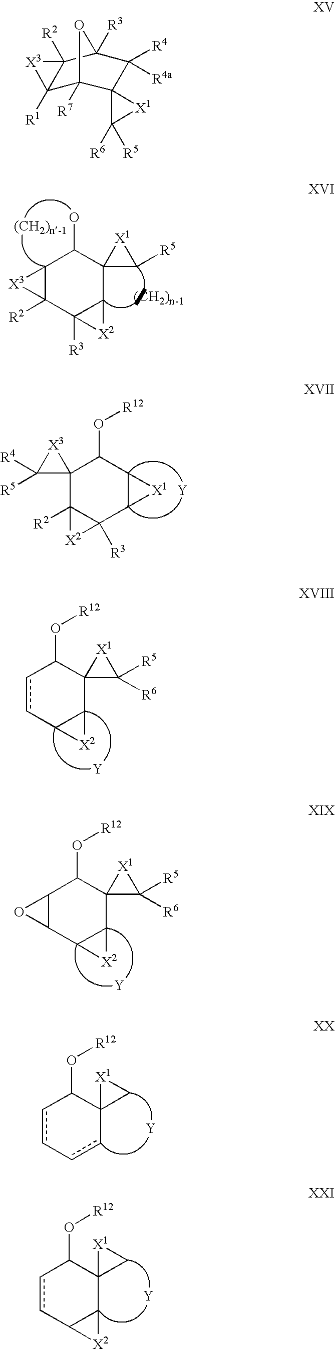

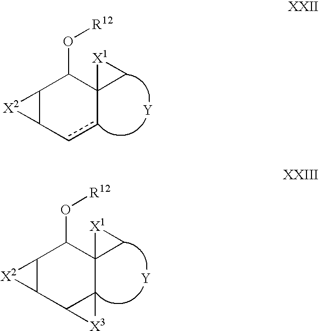

or a solvate, hydrate or pharmaceutically acceptable salt thereof; wherein:

(a) each dotted line indicates the presence of either a single bond or double bond, wherein the valences of a single bond are completed by hydrogens;

(b) X, X¹, X², and X³ are independently O, S, or NR¹¹, where R¹¹ is hydrogen or alkyl;

(c) R¹, R², R³, R⁴, R^{4a}, R⁵, R⁶, R⁷, R⁸, R⁹, and R¹⁰ are independently hydrogen or alkyl;

(d) R¹² is hydrogen, alkyl or carboxylalkyl; or R¹² is absent (when R² is absent, the O forms a carbonyl group with the carbon ring atom to which it is attached);

(e) Y, Y¹, Y², and Y³ each represents, together with the two or three ring carbon atoms to which each is attached, a 5- to 7-membered monocyclic carbon ring which can be saturated, partially saturated, or aromatic; and

(f) n, n′, and n″ are independently from 3 to 5.

Compounds within the scope of the invention include those in which:

X, X¹, X², and X³ are independently O, S, or NR¹¹, where R¹¹ is hydrogen or C₁₋₄ alkyl;

R¹, R², R³, R⁴, R^{4a}, R⁵, R⁶, R⁷, R⁸, R⁹, and R¹⁰ are independently hydrogen or C₁₋₄ alkyl;

Y, Y¹, Y², and Y³ each represents, together with the two or three ring carbon atoms to which each is attached, a C₅₋₇ cycloalkyl ring, a C₅₋₇ cycloalkenyl ring, or phenyl;

R¹² is hydrogen, C₁₋₄ alkyl or carboxyl(C₁₋₄)alkyl; or R¹² is absent; and

n, n′, and n″ are each from 3 to 5.

In one embodiment, the invention encompasses compounds of Formula I:

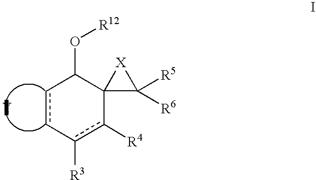

or a solvate, hydrate, prodrug or pharmaceutically acceptable salt thereof;

wherein:

(a) each dotted line indicates the presence of either a single bond or double bond, wherein the valences of a single bond are completed by hydrogens;

(b) X is O, S, or NR¹¹, where R¹¹ is hydrogen or alkyl;

(c) R³, R⁴, R⁵, and R⁶ are independently hydrogen or alkyl;

(d) R¹² is hydrogen, alkyl, carboxylalkyl, or R¹² is absent; and

(e) Y, together with the two ring carbon atoms to which it is attached, represents a 5- to 7-membered monocyclic carbon ring which is saturated, partially saturated, or aromatic.

Preferred compounds of Formula I are those for which X is O. Also preferred and those compounds in which R³, R⁴, R⁵, and R⁶ are each hydrogen. Also preferred are those compounds in which R¹² is absent.

Also preferred are those compounds of Formula I for which Y is phenyl or cyclohexyl. Compounds of Formula I in which Y is phenyl are especially preferred.

In a second embodiment, the invention encompasses compounds of Formula II:

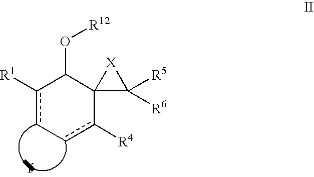

or a solvate, hydrate, prodrug or pharmaceutically acceptable salt thereof; wherein:

(a) the dotted line indicates the presence of either a single bond or double bond, wherein the valences of a single bond are completed by hydrogens;

(b) X is O, S, or NR¹¹, where R¹¹ is hydrogen or alkyl;

(c) R¹, R⁴, R⁵, and R⁶ are independently hydrogen or alkyl;

(d) R¹² is hydrogen, alkyl, carboxylalkyl, or R¹² is absent; and

(e) Y, together with the two ring carbon atoms to which it is attached, represents a 5- to 7-membered monocyclic carbon ring which can be saturated, partially saturated, or aromatic.

Preferred compounds of Formula II include those in which X is O. Also preferred are those compounds in which R¹² is absent.

Also preferred are those compounds in which R¹, R⁴, R⁵, and R⁶ are each hydrogen, especially those compounds in which X is O.

Preferred compounds of Formula II also include those in which Y is phenyl, particularly those in which X is O.

In a third embodiment, the present invention encompasses compounds of Formula III:

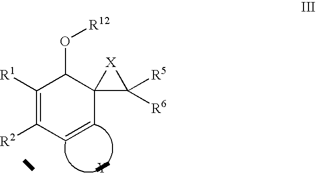

or a solvate, hydrate, prodrug or pharmaceutically acceptable salt thereof; wherein:

(a) X is O, S, or NR¹¹, where R¹¹ is hydrogen or alkyl;

(b) R¹, R², R⁵, and R⁶ are independently hydrogen or alkyl;

(c) R¹² is hydrogen, alkyl, carboxylalkyl, or R¹² is absent; and

(d) Y, together with the two ring carbon atoms to which it is attached, represents a 5- to 7-membered monocyclic carbon ring which can be saturated, partially saturated, or aromatic.

Preferred compounds of Formula III are those in which X is O. Also preferred are those compounds in which R¹² is absent.

Also preferred are those compounds in which R¹, R², R⁵, and R⁶ are each hydrogen, especially those compounds in which X is O.

Preferred compounds of Formula III also include those in which Y is phenyl, particularly those compounds in which X is O.

In a fourth embodiment, the invention encompasses those compounds of Formula IV:

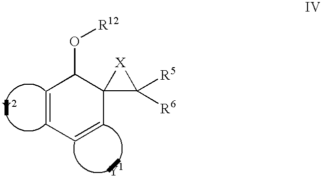

or a solvate, hydrate, prodrug or pharmaceutically acceptable salt thereof;

wherein:

(a) X is O, S, or NR¹¹, where R¹¹ is hydrogen or alkyl;

(b) R⁵ and R⁶ are independently hydrogen or alkyl;

(c) R¹² is hydrogen, alkyl, carboxylalkyl, or R¹² is absent; and

(d) Y¹ and Y² each represent, together with the two ring carbon atoms to which each is attached, a 5- to 7-membered monocyclic carbon ring which can be saturated, partially saturated, or aromatic.

Preferred compounds of Formula IV are those in which X is O. Also preferred are those compounds in which R¹² is absent.

Also preferred are those compounds in which R⁵ and R⁶ are each hydrogen, especially those compounds in which X is O.

Preferred compounds of Formula IV also include those in which Y¹ and Y² are both phenyl, particularly those compounds in which X is O.

In a fifth embodiment, the invention encompasses compounds of Formula V:

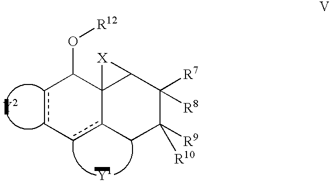

or a solvate, hydrate, prodrug or pharmaceutically acceptable salt thereof; wherein:

(a) the dotted line indicates the presence of either a single bond or double bond, wherein the valences of a single bond are completed by hydrogens;

(b) X is O, S, or NR¹¹, where R¹¹ is hydrogen or alkyl;

(c) R⁵, R⁷, R⁸, R⁹, and R¹⁰ are independently hydrogen or alkyl;

(d) R¹² is hydrogen, alkyl, carboxylalkyl, or R¹² is absent;

(e) Y¹ represents, together with the two ring carbon atoms to which it is attached, a 5- to 7-membered monocyclic carbon ring which can be saturated, partially saturated, or aromatic; and

(f) Y² represents, together with the three ring carbon atoms to which it is attached, a 5- to 7-membered monocyclic carbon ring which can be saturated, partially saturated, or aromatic.

Preferred compounds of Formula V includes those in which X is O. Also preferred are those compounds in which R¹² is absent.

Preferred compounds of Formula V also include those in which Y¹ and Y² are phenyl. Especially preferred are those compounds in which X is O and both Y¹ and Y² are phenyl. Also preferred are those compounds of Formula V in which R⁵, R⁷, R¹, R⁹, and R¹⁰ are each hydrogen.

In a sixth embodiment, the invention encompasses compounds of Formula VI:

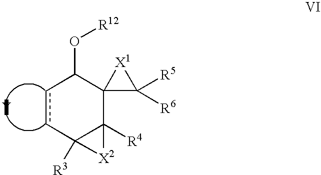

or a solvate, hydrate, prodrug or pharmaceutically acceptable salt thereof; wherein:

(a) the dotted line indicates the presence of either a single bond or double bond, wherein the valences of a single bond are completed by hydrogens;

(b) X¹ and X² are independently O, S, or NR¹¹, where R¹¹ is hydrogen or alkyl;

(c) R³, R⁴, R¹ and R⁶ are independently hydrogen or alkyl;

(d) R¹² is hydrogen, alkyl, carboxylalkyl, or R¹² is absent; and

(e) Y, together with the two ring carbon atoms to which it is attached, represents a 5- to 7-membered monocyclic carbon ring which can be saturated, partially saturated, or aromatic.

Preferred compounds of Formula VI are those in which X¹ and X² are independently O. Also preferred are those compounds in which R¹² is absent.

Also preferred are those compounds of Formula VI in which Y is phenyl or cyclohexyl. Particularly preferred are those compounds in which Y is phenyl and X is O.

Preferred compounds of Formula VI also include those compounds in which R³, R⁴, R⁵ and R⁶ are each hydrogen, especially those in which X¹ and X² are also both O.

In a seventh embodiment, the present invention encompasses compounds of Formula VII:

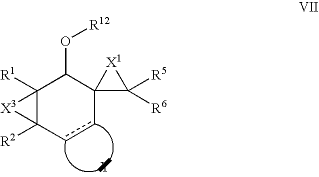

or a solvate, hydrate, prodrug or pharmaceutically acceptable salt thereof;

wherein:

(a) X¹ and X³ are independently O, S, or NR¹¹, where R¹¹ is hydrogen or alkyl;

(b) R¹, R², R⁵, and R⁶ are independently hydrogen or alkyl;

(c) R¹² is hydrogen, alkyl, carboxylalkyl, or R¹² is absent; and

(d) Y, together with the two ring carbon atoms to which it is attached, represents a 5- to 7-membered monocyclic carbon ring which can be saturated, partially saturated, or aromatic.

Preferred compounds of Formula VII are those in which X¹ and X³ are both O. Also preferred are those compounds in which R¹² is absent.

Also preferred are those compounds of Formula VII in which Y is phenyl. Compounds in which R¹, R², R⁵, and R⁶ are each hydrogen are also preferred, especially those in which R¹, R², R⁵, and R⁶ are each hydrogen and X¹ and X³ are each O.

In an eighth embodiment, the invention encompasses compounds of Formula VIII:

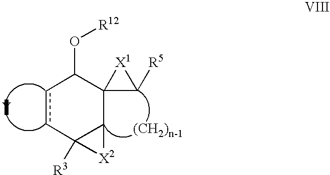

or a solvate, hydrate, prodrug or pharmaceutically acceptable salt thereof, wherein:

(a) the dotted line indicates the presence of either a single bond or double bond, wherein the valences of a single bond are completed by hydrogens;

(b) X¹ and X² are independently O, S, or NR¹¹, where R¹¹ is hydrogen or alkyl;

(c) R³ and R⁵ are independently hydrogen or alkyl;

(d) R¹² is hydrogen, alkyl, carboxylalkyl, or R¹² is absent;

(e) Y, together with the two ring carbon atoms to which it is attached, represents a 5- to 7-membered monocyclic carbon ring which can be saturated, partially saturated, or aromatic; and

(f) n is from 3 to 5.

Preferred compounds of Formula VIII are those in which X¹ and X² are both O. Also preferred are those compounds in which R¹² is absent.

Also preferred are those compounds of Formula VIII in which Y is phenyl. Compounds in which R³ and R⁵ are each hydrogen are also preferred, especially those in which R³ and R⁵ are each hydrogen, X¹ and X² are each O, and Y is phenyl.

Preferred compounds of Formula VIII also include those for which n has a value of 3 or 4, especially those in which n has the value of 4.

In a ninth embodiment, the invention encompasses compounds of Formula IX:

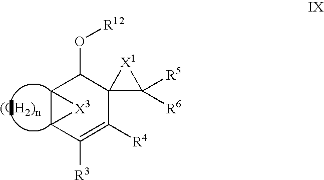

or a solvate, hydrate, prodrug or pharmaceutically acceptable salt thereof; wherein:

(a) X¹ and X³ are independently O, S, or NR¹¹, where R¹¹ is hydrogen or alkyl;

(b) R³, R⁴, R⁵, and R⁶ are independently hydrogen or alkyl;

(c) R¹² is hydrogen, alkyl, carboxylalkyl, or R¹² is absent; and

(d) n is from 3 to 5.

Preferred compounds of Formula IX are those in which X¹ and X³ are both O. Also preferred are those compounds in which R¹² is absent.

Also preferred are those compounds of Formula IX in which n is 3 or 4. Compounds in which n is 4 are particularly preferred.

Compounds in which R³, R⁴, R⁵, and R⁶ are each hydrogen are also preferred, especially those in which R³, R⁴, R⁵, and R⁶ are each hydrogen and X¹ and X³ are each O.

In a tenth embodiment, the present invention encompasses compounds of Formula X:

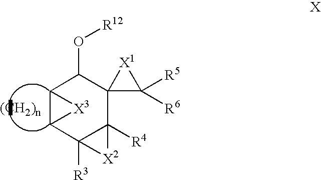

or a solvate, hydrate, prodrug or pharmaceutically acceptable salt thereof; wherein:

(a) X¹, X², and X³ are independently O, S, or NR¹¹, where R¹¹ is hydrogen or alkyl;

(b) R³, R⁴, R⁵, and R⁶ are independently hydrogen or alkyl;

(c) R¹² is hydrogen, alkyl, carboxylalkyl, or R¹² is absent; and

(d) n is from 3 to 5.

Preferred compounds of Formula X are those in which X¹, X², and X³ are each O. Also preferred are those compounds in which R¹² is absent.

Also preferred are those compounds of Formula X in which n is 3 or 4. Compounds in which n is 4 are particularly preferred.

Compounds in which R³, R⁴, R⁵, and R⁶ are each hydrogen are also preferred, especially those in which R³, R⁴, R⁵, and R⁶ are each hydrogen and X¹, X², and X³ are each O.

In an eleventh embodiment, the present invention encompasses compounds of Formula XI:

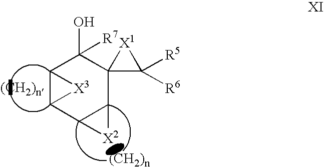

or a solvate, hydrate, prodrug or pharmaceutically acceptable salt thereof;

wherein:

(a) X¹, X², and X³ are independently O, S, or NR¹¹, where R¹¹ is hydrogen or alkyl;

(b) R⁵, R⁶, and R⁷ are independently hydrogen or alkyl; and

(c) n and n′ are each from 3 to 5.

Preferred compounds of Formula XI are those in which X¹, X², and X³ are each O.

Also preferred are those compounds in which n and n′ independently have a value of 3 or 4. Compounds of Formula XI in which both n and n′ are 4 are particularly preferred.

Compounds in which R⁵, R⁶, and R⁷ are each hydrogen are also preferred, especially those in which R⁵, R⁶, and R⁷ are each hydrogen and X¹, X², and X³ are each O.

In a twelfth embodiment, the present invention encompasses compounds of Formula XII:

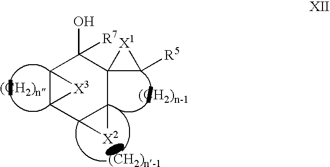

or a solvate, hydrate, prodrug or pharmaceutically acceptable salt thereof; wherein:

(a) X¹, X², and X³ are independently O, S, or NR¹¹, where R¹¹ is hydrogen or alkyl;

(b) R⁵ and R⁷ are independently hydrogen or alkyl; and

(c) n, n′ and n″ are each from 3 to 5.

Preferred compounds of Formula XII are those in which X¹, X², and X³ are each O.

Also preferred are those compounds in which n, n′ and n″ independently have a value of 3 or 4. Compounds of Formula XII in which both n, n′ and n″ are 4 are particularly preferred.

Compounds in which R¹ and R⁷ are each hydrogen are also preferred, especially those in which R⁵ and R⁷ are each hydrogen and X¹, X², and X³ are each O.

In a thirteenth embodiment, the present invention encompasses compounds of Formula XIII:

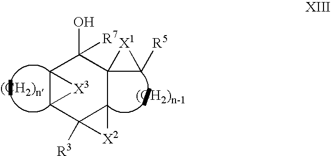

or a solvate, hydrate, prodrug or pharmaceutically acceptable salt thereof; wherein:

(a) X¹, X², and X³ are independently O, S, or NR¹¹, where R¹¹ is hydrogen or alkyl;

(b) R³, R⁵, and R⁷ are independently hydrogen or alkyl; and

(c) n and n′ are each from 3 to 5.

Preferred compounds of Formula XIII are those in which X¹, X², and X³ are each O.

Also preferred are those compounds of Formula XIII in which n and n′ independently have a value of 3 or 4. Compounds in which both n and n′ are 4 are particularly preferred.

Compounds in which R³, R⁵, and R⁷ are each hydrogen are also preferred, especially those in which R³, R⁵, and R⁷ are each hydrogen and X¹, X², and X³ are each O.

In a fourteenth embodiment, the invention also encompasses compounds of Formula XIV:

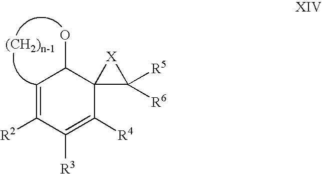

or a solvate, hydrate, prodrug or pharmaceutically acceptable salt thereof; wherein:

(a) X is O, S, or NR¹¹, where R¹¹ is hydrogen or alkyl;

(b) R², R³, R⁴, R⁵, and R⁶ are independently hydrogen or alkyl; and

(c) n is from 3 to 5.

Preferred compounds of Formula XIV are those in which X is O.

Also preferred are those compounds of Formula XIV in which n has a value of 3 or 4. Compounds in which n is 4 are particularly preferred.

Compounds in which R², R³, R⁴, R⁵, and R⁶ are each hydrogen are also preferred, especially those in which R², R³, R⁴, R⁵, and R⁶ are each hydrogen and X is O.

In a fifteenth embodiment, the present invention encompasses compounds of Formula XV:

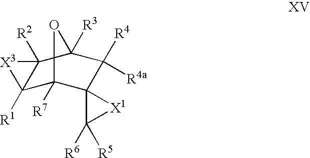

or a solvate, hydrate, prodrug or pharmaceutically acceptable salt thereof; wherein:

(a) X¹ and X³ are independently O, S, or NR¹¹, where R¹¹ is hydrogen or alkyl; and

(b) R¹, R², R³, R⁴, R^{4a}, R⁵, R⁶, and R⁷ are independently hydrogen or alkyl.

Preferred compounds of Formula XV are those in which X¹ and X³ are both O. Also preferred are those compounds of Formula XV in which R¹, R², R³, R⁴, R^{4a}, R⁵, R⁶, and R⁷ are each hydrogen.

In a sixteenth embodiment, the present invention encompasses compounds of Formula XVI:

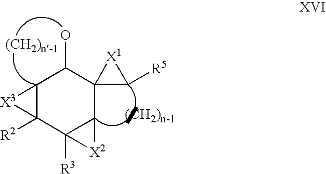

or a solvate, hydrate, prodrug or pharmaceutically acceptable salt thereof; wherein:

(a) X¹, X², and X³ are independently O, S, or NR¹¹, where R¹¹ is hydrogen or alkyl;

(b) R², R³, and R⁵ are independently hydrogen or alkyl; and

(c) n and n′ are each from 3 to 5.

Preferred compounds of Formula XVI are those in which X¹, X², and X³ are each O.

Also preferred are those compounds of Formula XVI in which n and n′ have a value of 3 or 4. Compounds in which n and n″ are both 4 are particularly preferred.

Compounds in which R², R³, and R⁵ are each hydrogen are also preferred, especially those in which R², R³, and R⁵ are each hydrogen and X¹, X², and X³ are each O.

In a seventeenth embodiment, the present invention encompasses compounds of Formula XVII:

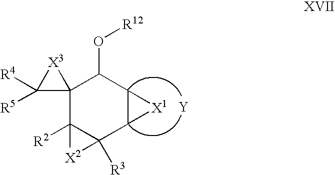

or a solvate, hydrate, prodrug or pharmaceutically acceptable salt thereof; wherein:

(a) X¹, X², and X³ are independently O, S, or NH;

(b) R², R³, R⁴, and R⁵ are independently hydrogen or alkyl;

(c) R¹² is hydrogen, alkyl, carboxylalkyl, or R¹² is absent; and

(d) Y, together with the two ring carbon atoms to which it is attached, represents a 5- to 7-membered monocyclic carbon ring which is saturated, or partially saturated.

Preferred compounds of Formula XVII are those in which X¹, X² and X³ are each O. Also preferred are those compounds in which R¹² is absent.

Also preferred are those compounds of Formula XVII in which Y is cyclohexyl. Compounds in which R², R³, R⁴, and R⁵ are each hydrogen are also preferred, especially those in which R², R³, R⁴, and R⁵ are each hydrogen and X¹, X² and X³ are each O.

In an eighteenth embodiment, the present invention encompasses compounds of Formula XVIII:

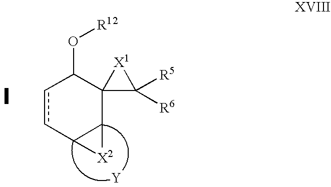

or a solvate, hydrate, prodrug or pharmaceutically acceptable salt thereof; wherein:

(a) the dotted line indicates the presence of either a single bond or double bond, wherein the valences of a single bond are completed by hydrogens;

(b) X¹ and X² are independently O, S, or NR¹¹, where R¹¹ is hydrogen or alkyl;

(c) R⁵ and R⁶ are independently hydrogen or alkyl;

(d) R¹² is hydrogen, alkyl, or carboxylalkyl, or R¹² is absent;

(e) Y represents, together with the two ring carbon atoms to which it is attached, a 5- to 7-membered monocyclic carbon ring which can be saturated, partially saturated, or aromatic.

Preferred compounds of Formula XVII are those in which X¹ and X² are each O. Also preferred are those compounds in which R¹² is absent.

Also preferred are those compounds of Formula XVIII in which Y is cyclohexyl. Compounds in which R⁵ and R⁶ are each hydrogen are also preferred, especially those in which R⁵ and R⁶ are each hydrogen and X¹ and X² are each O.

In a nineteenth embodiment, the present invention encompasses compounds of Formula XIX:

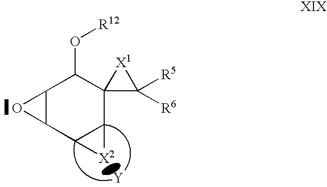

or a solvate, hydrate, prodrug or pharmaceutically acceptable salt thereof; wherein:

(a) X¹ and X² are independently O, S, or NR¹¹, where R¹¹ is hydrogen or alkyl;

(c) R⁵ and R⁶ are independently hydrogen or alkyl;

(d) R¹² is hydrogen, alkyl, or carboxylalkyl, or R¹² is absent;

(e) Y represents, together with the two ring carbon atoms to which it is attached, a 5- to 7-membered monocyclic carbon ring which can be saturated, partially saturated, or aromatic.

Preferred compounds of Formula XIX are those in which X¹ and X² are each O. Also preferred are those compounds in which R¹² is absent.

Also preferred are those compounds of Formula XIX in which Y is cyclohexyl. Compounds in which R⁵ and R⁶ are each hydrogen are also preferred, especially those in which R⁵ and R⁶ are each hydrogen and X¹ and X² are each O.

In a twentieth embodiment, the present invention encompasses compounds of Formula XX:

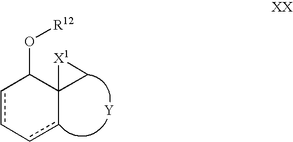

or a solvate, hydrate, prodrug or pharmaceutically acceptable salt thereof; wherein:

(a) each dotted line indicates the presence of either a single bond or double bond, wherein the valences of a single bond are completed by hydrogens;

(b) X¹ is O, S, or NR¹¹, where R¹¹ is hydrogen or alkyl;

(c) R¹² is hydrogen, alkyl, or carboxylalkyl, or R¹² is absent; and

(e) Y represents, together with the three ring carbon atoms to which it is attached, a 5- to 7-membered monocyclic carbon ring which can be saturated, partially saturated, or aromatic.

Preferred compounds of Formula XX are those in which X¹ is O. Also preferred are those compounds in which R¹² is absent.

Also preferred are those compounds of Formula XX in which Y is cyclohexyl.

In a twenty-first embodiment, the present invention encompasses compounds of Formula XXI:

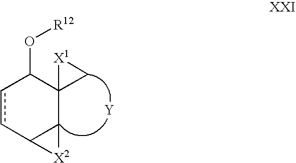

or a solvate, hydrate, prodrug or pharmaceutically acceptable salt thereof; wherein:

(a) a dotted line indicates the presence of either a single bond or double bond, wherein the valences of a single bond are completed by hydrogens;

(b) X¹ and X² are independently O, S, or NR¹¹, where R¹¹ is hydrogen or alkyl;

(c) R¹² is hydrogen, alkyl, or carboxylalkyl, or R¹² is absent; and

(d) Y represents, together with the three ring carbon atoms to which it is attached, a 5- to 7-membered monocyclic carbon ring which can be saturated, partially saturated, or aromatic.

Preferred compounds of Formula XXI are those in which X¹ and X² are both O. Also preferred are those compounds in which R¹² is absent.

Also preferred are those compounds of Formula XXI in which Y is cyclohexyl.

In a twenty-second embodiment, the present invention encompasses compounds of Formula XXII:

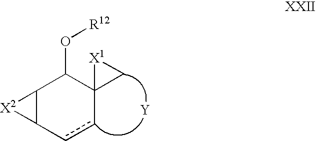

or a solvate, hydrate, prodrug or pharmaceutically acceptable salt thereof; wherein:

(a) a dotted line indicates the presence of either a single bond or double bond, wherein the valences of a single bond are completed by hydrogens;

(b) X¹ and X² are independently O, S, or NR¹¹, where R¹¹ is hydrogen or alkyl;

(c) R¹² is hydrogen, alkyl, or carboxylalkyl, or R¹² is absent; and

(d) Y represents, together with the three ring carbon atoms to which it is attached, a 5- to 7-membered monocyclic carbon ring which can be saturated, partially saturated, or aromatic.

Preferred compounds of Formula XXII are those in which X¹ and X² are both O. Also preferred are those compounds in which R¹² is absent.

Also preferred are those compounds of Formula XXII in which Y is cyclohexyl.

In a twenty-third embodiment, the present invention encompasses compounds of Formula XXIII:

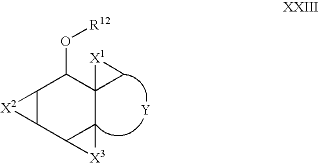

or a solvate, hydrate, prodrug or pharmaceutically acceptable salt thereof; wherein:

(a) X¹, X², and X³ are independently O, S, or NR¹¹, where R¹¹ is hydrogen or alkyl;

(c) R¹² is hydrogen, alkyl, or carboxylalkyl, or R¹² is absent; and

(d) Y represents, together with the three ring carbon atoms to which it is attached, a 5- to 7-membered monocyclic carbon ring which can be saturated, partially saturated, or aromatic.

Preferred compounds of Formula XXIII are those in which X¹, X², and X³ are each O. Also preferred are those compounds in which R¹² is absent.

Also preferred are those compounds of Formula XXIII in which Y is cyclohexyl.

Compounds of the present invention also include triptolide analogue compounds of Formula XXIII:

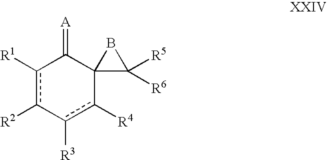

or its pharmaceutically acceptable salt or prodrug thereof, wherein:

(a) The dotted line indicates the presence of either a single or double bond, wherein the valences of a single bond are completed by hydrogens;

(b) A and B are independently O, S, NR⁷ or CR⁷R⁸;

(c) R¹, R², R³, R⁴, R⁵ and R⁶ are independently hydrogen, alkyl, alkenyl, alkynyl, cycloalkyl, cycloalkenyl, cycloalkynyl, aryl, alkaryl, arylalkyl, heterocyclic, heteroaryl, heteroaromatic, alkcarbonyl, carbonyl, carboxylic acid, ester, carbamate, amide, amine, hydroxyl, alkoxide, nitro, cyano, azide, sulfonyl, sulfanyl, sulfinyl, sulfamonyl, phosphonyl, phosphinyl, phosphoryl, phosphine, a residue of a natural or synthetic amino acid, a residue of a natural or synthetic carbohydrate or XR⁹ (wherein X═O, S or NR¹⁰), or

alternatively, one or more of R¹ and R², R² and R³, R³ and R⁴, R⁴ and R⁵, or R⁵ and R⁶, come together to form a bridged compound, preferably as a 3, 5, 6 or 7 membered ring, to form a cycloalkyl, cycloalkenyl, cycloalkynyl, aryl, heterocyclic, heteroaryl or heteroaromatic; and

(d) each R⁷, R⁸, R⁹ and R¹⁰ is independently hydrogen, alkyl, alkenyl, alkynyl, cycloalkyl, cycloalkenyl, cycloalkynyl, aryl, alkaryl, arylalkyl, heterocyclic, heteroaryl, heteroaromatic, alkcarbonyl, a residue of a natural or synthetic amino acid or a residue of a natural or synthetic carbohydrate.

Exemplary structures of compounds within the scope of the invention include the following:

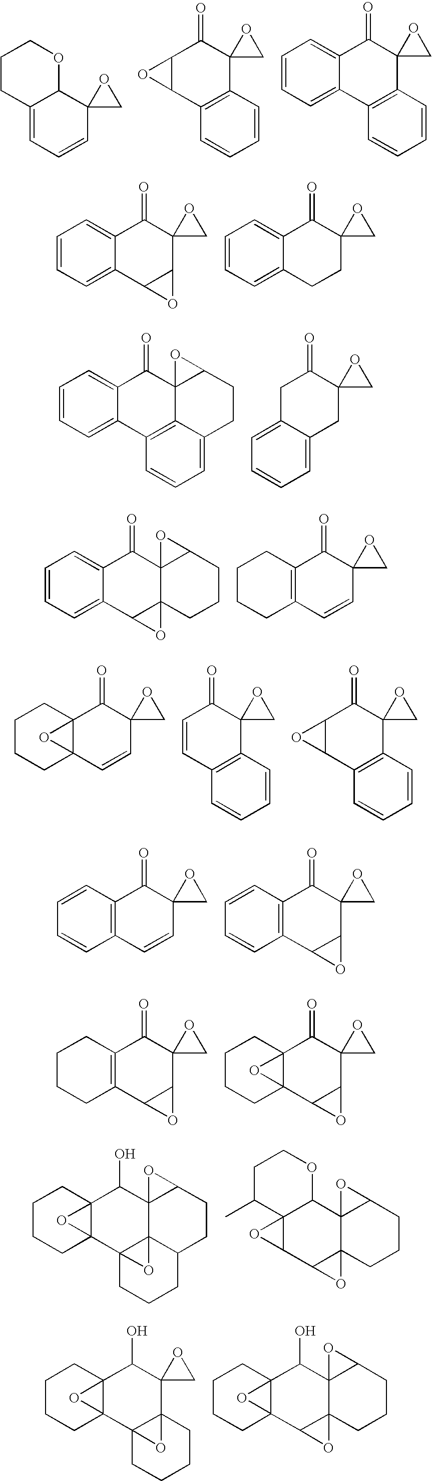

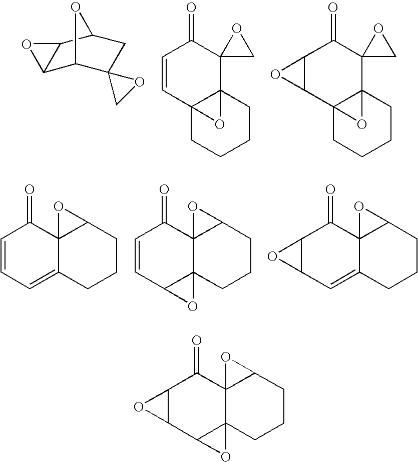

as well as pharmaceutically acceptable salts thereof.

It is also to be understood that the present invention is considered to include stereoisomers as well as optical isomers, e.g. mixtures of enantiomers as well as individual enantiomers and diastereomers, which arise as a consequence of structural asymmetry in selected compounds of the present series. The compounds of the present invention may also have polymorphic crystalline forms, with all polymorphic crystalline forms being included in the present invention.

The compounds of Formulae I-XXIV may also be solvated, especially hydrated. Hydration may occur during manufacturing of the compounds or compositions comprising the compounds, or the hydration may occur over time due to the hygroscopic nature of the compounds.

When any variable occurs more than one time in any constituent or in any one of Formulae I-XXIV, its definition on each occurrence is independent of its definition at every other occurrence. Also, combinations of substituents and/or variables are permissible only if such combinations result in stable compounds.

The term “alkyl” as employed herein by itself or as part of another group refers to both straight and branched chain radicals of up to 12 carbons, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl. Preferably, alkyl is 1 to 6 carbon atoms.

The term “cycloalkyl” as employed herein refers to cycloalkyl groups containing 5 to 7 carbon atoms. Typical examples include cyclopentyl, cyclohexyl, and cycloheptyl.

The term “cycloalkenyl” as employed herein refers to cycloalkenyl groups containing 5 to 7 carbon atoms, which can have one or more double bonds. Typical examples include cyclopentenyl, cyclohexenyl, 1,3-cyclopentadiene, 1,3-cyclohexadiene, 1,4-cyclohexadiene, and the like.

The term “carboxylalkyl” as employed herein refers to any of the above alkyl groups substituted by one or more carboxylic acid moieties.

The phrase “a 5- to 7-membered monocyclic carbon ring which can be saturated, partially unsaturated, or aromatic” as employed herein refers to cycloalkyl and cycloalkenyl groups, as defined herein, and phenyl.

The term “independently” is used herein to indicate that the variable that is independently applied varies independently from application to application. Thus, in a compound such as R“XYR”, wherein R¹¹ is “independently carbon or nitrogen,” both R¹¹ can be carbon, both R¹¹ can be nitrogen, or one R¹¹ can be carbon and the other R¹¹ nitrogen.

The term alkyl, as used herein for compounds of Formula XXIV, unless otherwise specified, refers to a saturated straight, branched, or cyclic, primary, secondary, or tertiary hydrocarbon, typically of C, to C₁₈ and specifically includes methyl, ethyl, propyl, isopropyl, butyl, isobutyl, t-butyl, pentyl, cyclopentyl, isopentyl, neopentyl, hexylisohexyl, cyclohexyl, cyclohexylmethyl, 3-methylpentyl, 2,2-dimethylbutyl and 2,3-dimethylbutyl. The alkyl group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thiol, imine, sulfonic acid, sulfate, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphate, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term alkylene or alkenyl for compounds of Formula XXIV refers to a saturated hydrocarbyldiyl radical of straight or branched configuration, including but not limited to those that have from one to ten carbon atoms. Included within the scope of this term are methylene, 1,2-ethane-diyl, 1,1-ethane-diyl, 1,3-propane-diyl, 1,2-propane-diyl, 1,3-butane-diyl, 1,4-butane-diyl and the like. The alkylene group or other divalent moiety disclosed herein can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term aryl, as used herein for compounds of Formula XXIV, and unless otherwise specified, refers to phenyl, biphenyl, or naphthyl, and preferably phenyl. The aryl group can be optionally substituted with one or more moieties selected from the group consisting of hydroxyl, amino, alkylamino, arylamino, alkoxy, aryloxy, halo, nitro, cyano, sulfonic acid, sulfate, phosphonic acid, phosphate or phosphonate, either unprotected, or protected as necessary, as known to those skilled in the art, for example, as taught in Greene, et al., “Protective Groups in Organic Synthesis,” John Wiley and Sons, Second Edition, 1991.

The term aralkyl, as used herein for compounds of Formula XXIV, and unless otherwise specified, refers to an aryl group as defined above linked to the molecule through an alkyl group as defined above. The term alkaryl or alkylaryl as used herein, and unless otherwise specified, refers to an alkyl group as defined above linked to the molecule through an aryl group as defined above. In each of these groups, the alkyl group can be optionally substituted as describe above and the aryl group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included within the scope of the term aryl are phenyl; naphthyl; phenylmethyl; phenylethyl; 3,4,5-trihydroxyphenyl; 3,4,5-trimethoxyphenyl; 3,4,5-triethoxyphenyl; 4-chlorophenyl; 4-methylphenyl; 3,5-di-tertiarybutyl-4-hydroxyphenyl; 4-fluorophenyl; 4-chloro-1-naphthyl; 2-methyl-1-naphthylmethyl; 2-naphthylmethyl; 4-chlorophenylmethyl; 4-tertiarybutylphenyl; 4-tertiarybutylphenylmethyl and the like.

The term alkoxy, as used herein for compounds of Formula XXIV, and unless otherwise specified, refers to a moiety of the structure —O-alkyl, wherein alkyl is as defined above.

The term heteroaryl or heteroaromatic, as used herein for compounds of Formula XXIV, refers to an aromatic that includes at least one sulfur, oxygen, nitrogen or phosphorus in the aromatic ring. The term heterocyclic refers to a nonaromatic cyclic group wherein there is at least one heteroatom, such as oxygen, sulfur, nitrogen or phosphorus in the ring. Nonlimiting examples of heteroaryl and heterocyclic groups include furyl, furanyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzimidazolyl, purinyl, carbazolyl, oxazolyl, thiazolyl, isothiazolyl, 1,2,4-thiadiazolyl, isooxazolyl, pyrrolyl, quinazolinyl, cinnolinyl, phthalazinyl, xanthinyl, hypoxanthinyl, thiophene, furan, pyrrole, isopyrrole, pyrazole, or imidazole. The heteroaromatic group can be optionally substituted as described above for aryl. The heterocyclic group can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference. The heteroaromatic can be partially or totally hydrogenated as desired. As a nonlimiting example, dihydropyridine can be used in place of pyridine. Functional oxygen and nitrogen groups on the heteroaryl group can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acyl groups such as acetyl and propionyl, methanesulfonyl, and p-toluenesulfonyl.

The term amino acid for compounds of Formula XXIV includes naturally occurring and synthetic amino acids, and includes but is not limited to, alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl and histidinyl.

The term “amide,” as used herein for compounds of Formula XXIV, refers to a carbonyl moiety wherein the non-alkyl moiety is formed from an amine. Some non-limiting examples are formylamino, acetylamino, propionylamino, butanoylamino, isobutanoylamino, pentanoylamino, 3-methyl-butanoylamino, hexanoylamino, methoxycarbonylamino, ethoxycarbonylamino, n-propoxycarbonylamino, isopropoxycarbonylamino, benzamido, cyclopentylcarbonyl-amido, cyclohexylcarbonylamido, cycloheptylcarbonyl-amido, phenylacetylamido, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “sulfamoyl” as used herein for compounds of Formula XXIV is a hexavalent sulfur covalently bound to at least two oxygens and a nitrogen. Some non-limiting examples include methanesulphonylamino, ethanesulphonylamino, n-propanesulphonylamino, isopropanesulphonylamino, n-butane-sulphonylamino, N-ethyl-phenylmethanesulphonylamido, N-ethyl-2-phcnylethane-sulphonylamido, N-ethyl-3-phenylpropanesulphonylamido, N-ethyl-naphthalen-1-yl-sulphonamido or N-ethyl-naphthalen-2-yl-sulphonylamido. The sulfamoyl group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “ester” as used herein for compounds of Formula XXIV refers to a carbonyl flanked by an alkoxy group and a carbon based group. Some non-limiting examples include hydroxycarbonyl, methoxycarbonyl, ethoxycarbonyl, n-propyloxycarbonyl, isopropyloxycarbonyl, n-butyloxycarbonyl, isobutyloxycarbonyl, tert-butyloxycarbonyl or 1-(cinnamyloxycarbonyloxy)-ethoxycarbonyl. The ester group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “urethane” or “carbamate” as used herein for compounds of Formula XXIV refers to —OC(O)NR⁴R⁵ in which R⁴ and R⁵ are independently selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C, to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the carbamide optimally comprise a phenyl group. The term “lower carbamide” refers to an carbamide group in which the non-carbonyl moiety is a lower alkyl. The carbamide group also can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term carbohydrate, used herein for compounds of Formula XXIV refers to mono, di, tri, oligo, and poly saccharides consisting of furanose and pyranose sugars such as threose, ribulose, ketose, gentiobiose, aldose, aldotetrose, aldopentose, aldohexose, ketohexose, ketotetrose, ketopentose, erythrose, threose, ribose, deoxyribose, arabinose, xylose, lyxose, allose, altrose, glucose, mannose, gulose, idose, glactose, talose, erythrulose, ribulose, xylulose, psicose, fructose, sorbose, tagatose, dextrose, maltose, lactose, sucrose, or cellulose. The carbohydrate moiety as disclosed herein can be optionally substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term alkylheteroaryl as used here for compounds of Formula XXIV refers to an alkyl group substituted by a heteroaryl substituent.

The term halo or halogen, as used herein includes chloro, bromo, iodo and fluor.

The term alkoxy, as used herein, and unless otherwise specified, refers to a moiety of the structure —O-alkyl, wherein alkyl is as defined above.

The compounds of the invention can be made according to Schemes 1-3, which outline a synthetic route to compounds of Formulae I-XXIV.

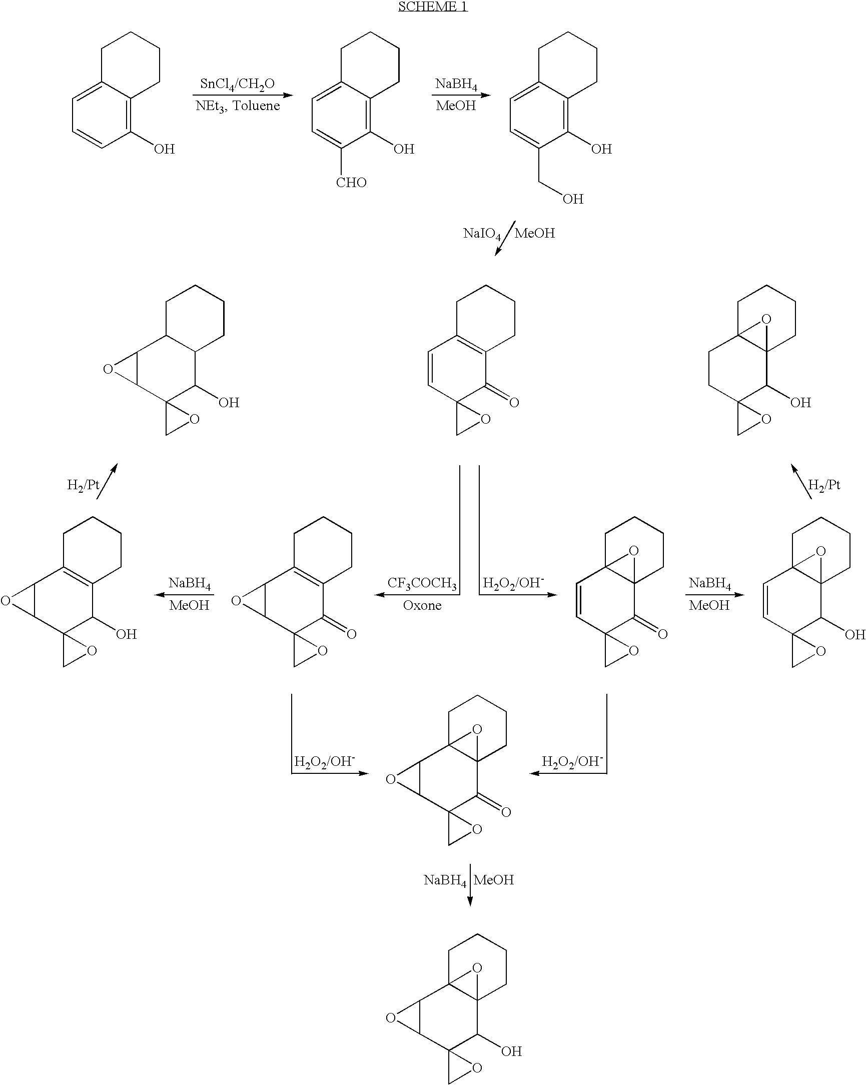

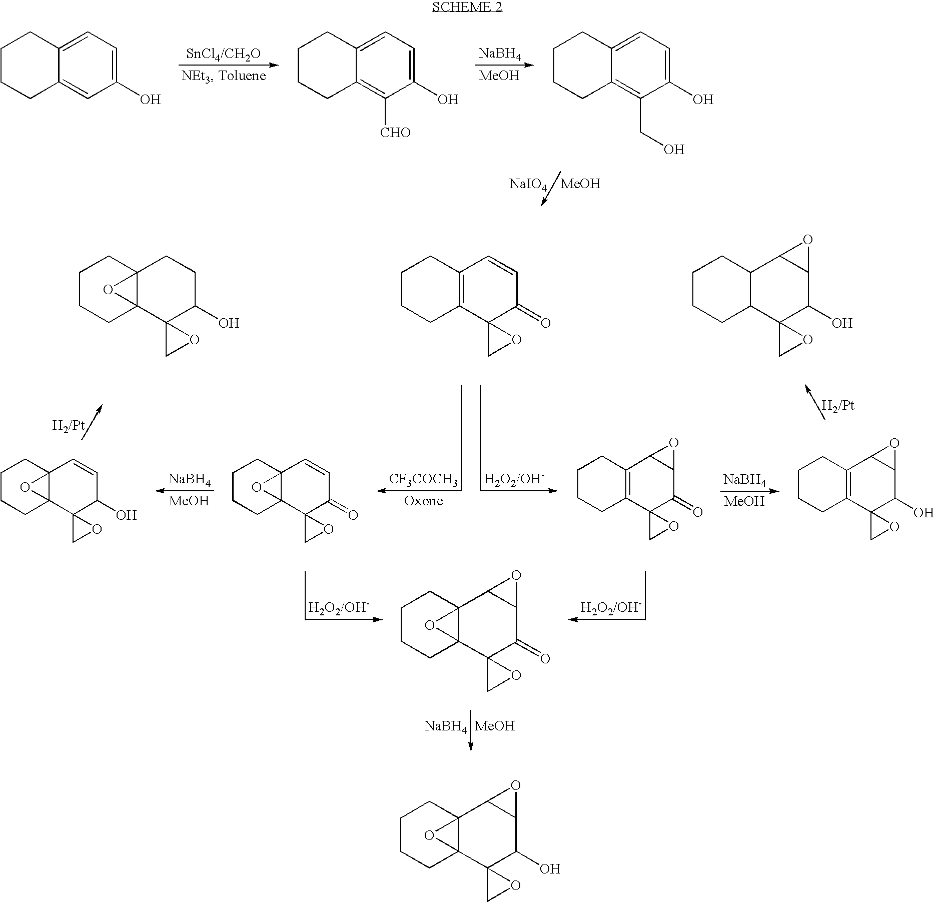

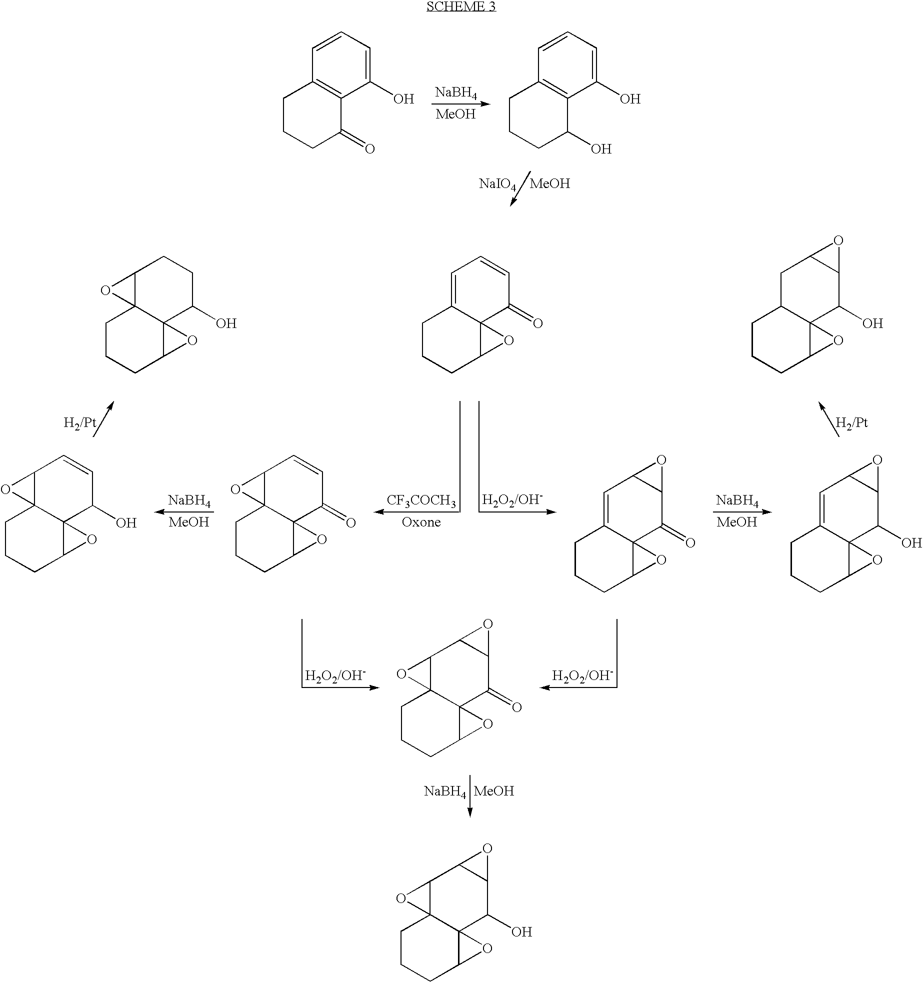

In cases where compounds of the invention are sufficiently basic or acidic to form stable nontoxic acid or base salts, administration of the compound as a pharmaceutically acceptable salt maybe appropriate. The term “pharmaceutically acceptable salts” or “complexes” refers to salts or complexes that retain the desired biological activity of the compounds of the present invention and exhibit minimal undesired toxicological effects.

Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts. Alternatively, the pharmaceutically acceptable salts may be made with sufficiently basic compounds such as an amine with a suitable acid affording a physiologically acceptable anion. Alkali metal (for example, sodium, potassium or lithium) or alkaline earth metal (for example calcium) salts of carboxylic acids can also be made.

Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like. Also included in this definition are pharmaceutically acceptable quaternary salts known by those skilled in the art, which specifically include the quaternary ammonium salt of the formula —NR⁺A⁻, wherein R is as defined above and A is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess anti-inflammatory activity, or are metabolized to a compound that exhibits such activity.

Any of the compounds described herein can be administered as a prodrug to increase the activity, bioavailability, stability or otherwise alter the properties of the compound. A number of prodrug ligands are known. In general, alkylation, acylation or other lipophilic modification of the compound will increase the stability of the compound. Examples of substituent groups that can replace one or more hydrogens on the compound are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed compounds to achieve a desired effect.

For their end-use application, the potency and other biochemical parameters of the compounds of the present invention are readily ascertained by standard biochemical techniques known to those of skill in the art. Actual dose ranges for their specific end-use application will, of course, depend upon the nature and severity of the disease state of the patient or animal to be treated, as determined by the attending diagnostician. It is expected that a useful dose range will be about 0.01 to 10 mg per kg per day for an effective therapeutic effect.

Compounds of the present invention possess potent antiinflammatory and immunsuppressive activities and may be employed for a number of therapeutic purposes. Thus, compounds of the invention may be used to treat the following disorders:

Autoimmune and Inflamatory Diseases: The compounds of the present invention can be used to treat a wide variety of autoimmune and inflammatory disease states, including but not limited to arthritis, asthma, dermatitis, psoriasis, cystic fibrosis, post transplantation late and chronic solid organ rejection, multiple sclerosis, systemic lupus erythematosis, inflammatory bowel diseases, autoimmune diabetes, diabetic retinopathy, rhinitis, ischemia-reperfusion injury, post-angioplasty restenosis, chronic obstructive pulmonary disease (COPD), glomerulonephritis, Graves disease, gastrointestinal allergies, and conjunctivitis.

Nonlimiting examples of arthritis include rheumatoid (such as soft-tissue rheumatism and non-articular rheumatism, fibromyalgia, fibrositis, muscular rheumatism, myofascil pain, humeral epicondylitis, frozen shoulder, Tietze's syndrome, fascitis, tendinitis, tenosynovitis, bursitis), juvenile chronic, spondyloarthropaties (ankylosing spondylitis), osteoarthritis, hyperuricemia and arthritis associated with acute gout, chronic gout and systemic lupus erythematosus.

Additional examples include human endothelial disorders such as psoriasis, eczematous dermatitis, Kaposi's sarcoma as well as proliferative disorders of smooth muscle cells.

In yet another embodiment, the compounds disclosed herein can be selected to treat anti-inflammatory conditions that are mediated by mononuclear leukocytes.

In one embodiment, the compounds of the present invention are selected for the prevention or treatment of tissue or organ transplant rejection. Treatment and prevention of organ or tissue transplant rejection includes, but are not limited to treatment of recipients of heart, lung, combined heart-lung, liver, kidney, pancreatic, skin, spleen, small bowel, or corneal transplants. The compounds can also be used in the prevention or treatment of graft-versus-host disease, such as sometimes occurs following bone marrow transplantation.

Asthma: In one embodiment, the compound of the present invention is administered in combination or alternation with heparin, frusemide, ranitidine, an agent that effects respiratory function, such as DNAase, or immunosuppressive agents, IV gamma globulin, troleandomycin, cyclosporin (Neoral), methotrexate, FK-506, gold compounds such as Myochrysine (gold sodium thiomalate), platelet activating factor (PAF) antagonists such as thromboxane inhibitors, leukotriene D₄-receptor antagonists such as Accolate (zafirlukast), Ziflo (zileuton), leukotriene C₁ or C₂ antagonists and inhibitors of leukotriene synthesis such as zileuton for the treatment of asthma, or an inducible nitric oxide synthase inhibitor.

In another embodiment, the active compound is administered in combination or alternation with one or more other prophylactic agent(s). Examples of prophylactic agents that can be used in alternation or combination therapy include but are not limited to sodium cromoglycate, Intal (cromolyn sodium, Nasalcrom, Opticrom, Crolom, Ophthalmic Crolom), Tilade (nedocromil, nedocromil sodium) and ketotifen.

In another embodiment, the active compound is administered in combination or alternation with one or more other β₂-adrenergic agonist(s) (β agonists). Examples of β₂-adrenergic agonists (β agonists) that can be used in alternation or combination therapy include but are not limited to albuterol (salbutamol, Proventil, Ventolin), terbutaline, Maxair (pirbuterol), Serevent (salmeterol), epinephrine, metaproterenol (Alupent, Metaprel), Brethine (Bricanyl, Brethaire, terbutaline sulfate), Tornalate (bitolterol), isoprenaline, ipratropium bromide, bambuterol hydrochloride, bitolterol meslyate, broxaterol, carbuterol hydrochloride, clenbuterol hydrochloride, clorprenaline hydrochloride, efirmoterol fumarate, ephedra (source of alkaloids), ephedrine (ephedrine hydrochloride, ephedrine sulfate), etafedrine hydrochloride, ethylnoradrenaline hydrochloride, fenoterol hydrochloride, hexoprenaline hydrochloride, isoetharine hydrochloride, isoprenaline, mabuterol, methoxyphenamine hydrochloride, methylephedrine hydrochloride, orciprenaline sulphate, phenylephrine acid tartrate, phenylpropanolamine (phenylpropanolamine polistirex, phenylpropanolamine sulphate), pirbuterol acetate, procaterol hydrochloride, protokylol hydrochloride, psuedoephedrine (psuedoephedrine polixtirex, psuedoephedrine tannate, psuedoephedrine hydrochloride, psuedoephedrine sulphate), reproterol hydrochloride, rimiterol hydrobromide, ritodrine hydrochloride, salmeterol xinafoate, terbutaline sulphate, tretoquinol hydrate and tulobuterol hydrochloride.

In another embodiment, the active compound is administered in combination or alternation with one or more other corticosteroid(s). Examples of corticosteriods that can be used in alternation or combination therapy include but are not limited to glucocorticoids (GC), Aerobid (Aerobid-M, flunisolide), Azmacort (triamcinolone acetonide), Beclovet (Vanceril, beclomethasone dipropionate), Flovent (fluticasone), Pulmicort (budesonide), prednisolone, hydrocortisone, adrenaline, Aldlometasone Dipropionate, Aldosterone, Amcinonide, Beclomethasone Dipropionate, Bendacort, Betamethasone (Betamethasone Acetate, Betamnethasone Benzoate, Betamethasone Dipropionate, Betamethasone Sodium Phosphate, Betamethasone Valerate), Budesonide, Ciclomethasone, Ciprocinonide, Clobetasol Propionate, Clobetasone Butyrate, Clocortolone Pivalate, Cloprednol, Cortisone Acetate, Cortivazol, Deflazacort, Deoxycortone Acetate (Deoxycortone Pivalate), Deprodone, Desonide, Desoxymethasone, Dexamethasone (Dexamethasone Acetate, Dexamethasone Isonicotinate, Dexamethasone Phosphate, Dexamethasone Sodium Metasulphobenzoate, Dexamethasone Sodium Phosphate), Dichlorisone Acetate, Diflorasone Diacetate, Diflucortolone Valerate, Difluprednate, Domoprednate, Endrysone, Fluazacort, Fluclorolone Acetonide, Fludrocortisone Acetate, Flumethasone (Flumethasone Pivalate), Flunisolide, Fluocinolone Acetonide, Fluocinonide, Fluocortin Butyl, Fluocortolone (Fluocortolone Hexanoate, Fluocortolone Pivalate), Fluorometholone (Fluorometholone Acetate), Fluprednidene Acetate, Fluprednisolone, Flurandrenolone, Fluticasone Propionate, Formocortal, Halcinonide, Halobetasol Propionate, Halometasone, Hydrocortamnate Hydrochloride, Hydrocortisone (Hydrocortisone Acetate, Hydrocortisone Butyrate, Hydrocortisone Cypionate, Hydrocortisone Hemisuccinate, Hydrocortisone Sodium Phosphate, Hydrocortisone Sodium Succinate, Hydrocortisone Valerate), Medrysone, Meprednisone, Methylprednisolone (Methylprednisolone Acetate, Methylprednisolone ,Hemisuccinate, Methylprednisolone Sodium Succinate), Mometasone Furoate, Paramethasone Acetate, Prednicarbate, Prednisolamate Hydrochloride, Prednisolone (Prednisolone Acetate, Prednisolone Hemisuccinate, Prednisolone Hexanoate, Prednisolone Pivalate, Prednisolone Sodium Metasulphobenzoate, Prednisolone Sodium Phosphate, Prednisolone Sodium Succinate, Prednisolone Steaglate, Prednisolone Tebutate), Prednisone (Prednisone Acetate), Prednylidene, Procinonide, Rimexolone, Suprarenal Cortex, Tixocortol Pivalate, Triamcinolone (Triamcinolone Acetonide, Triamcinolone Diacetate and Triamcinolone Hexacetonide).

In another embodiment, the active compound is administered in combination or alternation with one or more other antihistamine(s) (H₁ receptor antagonists). Examples of antihistamines (H₁ receptor antagonists) that can be used in alternation or combination therapy include alkylamines, ethanolamines, ethylenediamines, piperazines, piperidines or phenothiazines. Some non-limiting examples of antihistamines are Chlortrimeton (Teldrin, chlorpheniramine), Atrohist (brompheniramine, Bromarest, Bromfed, Dimetane), Actidil (triprolidine), Dexchlor (Poladex, Polaramine, dexchlorpheniramine), Benadryl (diphen-hydramine), Tavist (clemastine), Dimetabs (dimenhydrinate, Dramamine, Marmine), PBZ (tripelennamine), pyrilamine, Marezine (cyclizine), Zyrtec (cetirizine), hydroxyzine, Antivert (meclizine, Bonine), Allegra (fexofenadine), Hismanal (astemizole), Claritin (loratadine), Seldane (terfenadine), Periactin (cyproheptadine), Nolamine (phenindamine, Nolahist), Phenameth (promethazine, Phenergan), Tacaryl (methdilazine) and Temaril (trimeprazine).

Alternatively, the compound of the present invention is administered in combination or alternation with the following: (a) xanthines and methylxanthines, such as Theo-24 (theophylline, Slo-Phylline, Uniphyllin, Slobid, Theo-Dur), Choledyl (oxitriphylline), aminophylline; (b) anticholinergic agents (antimuscarinic agents) such as belladonna alkaloids, Atrovent (ipratropium bromide), atropine, oxitropium bromide; (c) phosphodiesterase inhibitors such as zardaverine; (d) calcium antagonists such as nifedipine; or (e) potassium activators such as cromakalim for the treatment of asthma.

Arthritic Disorders: In one embodiment, the compound of the present invention can also be administered in combination or alternation with apazone, amitriptyline, chymopapain, collegenase, cyclobenzaprine, diazepam, fluoxetine, pyridoxine, ademetionine, diacerein, glucosamine, hylan (hyaluronate), misoprostol, paracetamol, superoxide dismutase mimics, TNFα receptor antagonists, TNFα antibodies, P38 Kinase inhibitors, tricyclic antidepressents, cJun kinase inhibitors or immunosuppressive agents, IV gamma globulin, troleandomycin, cyclosporin (Neoral), methotrexate, FK-506, gold compounds such as Myochrysine (gold sodium thiomalate), platelet activating factor (PAF) antagonists such as thromboxane inhibitors, leukotriene-D₄-receptor antagonists such as Accolate (zafirlukast), Ziflo (zileuton), leukotriene C₁, C₂ antagonists and inhibitors of leukotriene synthesis such as zileuton for the treatment of arthritic disorders, inducible nitric oxide sythase inhibitors.

In another embodiment, the active compound is administered in combination or alternation with one or more other corticosteriod(s). Examples of corticosteriods that can be used in alternation or combination therapy include but are not limited to glucocorticoids (GC), Aerobid (Aerobid-M, flunisolide), Azmacort (triamcinolone acetonide), Beclovet (Vanceril, beclomethasone dipropionate), Flovent (fluticasone), Pulmicort (budesonide), prednisolone, hydrocortisone, adrenaline, Alclometasone Dipropionate, Aldosterone, Ameinonide, Beclomethasone Dipropionate, Bendacort, Betamethasone (Betamethasone Acetate, Betamethasone Benzoate, Betamethasone Dipropionate, Betamethasone Sodium Phosphate, Betamethasone Valerate), Budesonide, Ciclomethasone, Ciprocinonide, Clobetasol Propionate, Clobetasone Butyrate, Clocortolone Pivalate, Cloprednol, Cortisone Acetate, Cortivazol, Deflazacort, Deoxycortone Acetate (Deoxycortone Pivalate), Deprodone, Desonide, Desoxymethasone, Dexamethasone (Dexamethasone Acetate, Dexamethasone Isonicotinate, Dexamethasone Phosphate, Dexamnethasone Sodium Metasulphobenzoate, Dexamethasone Sodium Phosphate), Dichlorisone Acetate, Diflorasone Diacetate, Diflucortolone Valerate, Difluprednate, Domoprednate, Endrysone, Fluazacort, Fluclorolone Acetonide, Fludrocortisone Acetate, Flumethasone (Flumethasone Pivalate), Flunisolide, Fluocinolone Acetonide, Fluocinonide, Fluocortin Butyl, Fluocortolone (Fluocortolone Hexanoate, Fluocortolone Pivalate), Fluorometholone (Fluorometholone Acetate), Fluprednidene Acetate, Fluprednisolone, Flurandrenolone, Fluticasone Propionate, Formocortal, Halcinonide, Halobetasol Propionate, Halometasone, Hydrocortamate Hydrochloride, Hydrocortisone (Hydrocortisone Acetate, Hydrocortisone Butyrate, Hydrocortisone Cypionate, Hydrocortisone Hemisuccinate, Hydrocortisone Sodium Phosphate, Hydrocortisone Sodium Succinate, Hydrocortisone Valerate), Medrysone, Meprednisone, Methylprednisolone (Methylprednisolone Acetate, Methylprednisolone ,Hemisuccinate, Methylprednisolone Sodium Succinate), Mometasone Furoate, Paramethasone Acetate, Prednicarbate, Prednisolamate Hydrochloride, Prednisolone (Prednisolone Acetate, Prednisolone Hemisuccinate, Prednisolone Hexanoate, Prednisolone Pivalate, Prednisolone Sodium Metasulphobenzoate, Prednisolone Sodium Phosphate, Prednisolone Sodium Succinate, Prednisolone Steaglate, Prednisolone Tebutate), Prednisone (Prednisone Acetate), Prednylidene, Procinonide, Rimexolone, Suprarenal Cortex, Tixocortol Pivalate, Triamcinolone (Triamcinolone Acetonide, Triamcinolone Diacetate and Triamcinolone Hexacetonide).

In another embodiment, the active compound is administered in combination or alternation with one or more other non-steroidal anti-inflammatory drug(s) (NSAIDS). Examples of NSAIDS that can be used in alternation or combination therapy are carboxylic acids, propionic acids, fenamates, acetic acids, pyrazolones, oxicans, alkanones, gold compounds and others that inhibit prostaglandin synthesis, preferably by selectively inhibiting cylcooxygenase-2 (COX-2). Some nonlimiting examples of COX-2 inhibitors are Celebrex (celecoxib) and Vioxx (rofacoxib). Some non-limiting examples of NSAIDS are aspirin (acetylsalicylic acid), Dolobid (diflunisal), Disalcid (salsalate, salicylsalicylate), Trisilate (choline magnesium trisalicylate), sodium salicylate, Cuprimine (penicillamine), Tolectin (tolmetin), ibuprofen (Motrin, Advil, Nuprin Rufen), Naprosyn (naproxen, Anaprox, naproxen sodium), Nalfon (fenoprofen), Orudis (ketoprofen), Ansaid (flurbiprofen), Daypro (oxaprozin), meclofenamate (meclofanamic acid, Meclomen), mefenamic acid, Indocin (indomethacin), Clinoril (sulindac), tolmetin, Voltaren (diclofenac), Lodine (etodolac), ketorolac, Butazolidin (phenylbutazone), Tandearil (oxyphenbutazone), piroxicam (Feldene), Relafen (nabumetone), Myochrysine (gold sodium thiomalate), Ridaura (auranofin), Solganal (aurothioglucose), acetaminophen, colchicine, Zyloprim (allopurinol), Benemid (probenecid), Anturane (sufinpyrizone), Plaquenil (hydroxychloroquine), Aceclofenac, Acemetacin, Acetanilide, Actarit, Alclofenac, Alminoprofen, Aloxiprin, Aluminium Aspirin, Amfenac Sodium, Amidopyrine, Aminopropylone, Ammonium Salicylate, Ampiroxicam, Amyl Salicylate, Anirolac, Aspirin, Auranofin, Aurothioglucose, Aurotioprol, Azapropazone, Bendazac (Bendazac Lysine), Benorylate, Benoxaprofen, Benzpiperylone, Benzydamine hydrochloride, Bomyl Salicylate, Bromfenac Sodium, Bufexamac, Bumadizone Calcium, Butibufen Sodium, Capsaicin, Carbaspirin Calcium, Carprofen, Chlorthenoxazin, Choline Magnesium Trisalicylate, Choline Salicylate, Cinmetacin, Clofexamide, Clofezone, Clometacin, Clonixin, Cloracetadol, Cymene, Diacerein, Diclofenac (Diclofenac Diethylammonium Salt, Diclofenac Potassium, Diclofenac Sodium), Diethylamine Salicylate, Diethylsalicylamide, Difenpiramide, Diflunisal, Dipyrone, Droxicam, Epirizole, Etenzamide, Etersalate, Ethyl Salicylate, Etodolac, Etofenamate, Felbinac, Fenbufen, Fenclofenac, Fenoprofen Calcium, Fentiazac, Fepradinol, Feprazone, Floctafenine, Flufenamic, Flunoxaprofen, Flurbiprofen (Flurbiprofen Sodium), Fosfosal, Furprofen, Glafenine, Glucametacin, Glycol Salicylate, Gold Keratinate, Harpagophytum Procumbens, Ibufenac, Ibuprofen, Ibuproxam, Imidazole Salicylate, Indomethacin (Indomethacin Sodium), Indoprofen, Isamifazone, Isonixin, Isoxicam, Kebuzone, Ketoprofen, Ketorolac Trometamol, Lithium Salicylate, Lonazolac Calcium, Lomoxicam, Loxoprofen Sodium, Lysine Aspirin, Magnesium Salicylate, Meclofenamae Sodium, Mefenamic Acid, Meloxicam, Methyl Butetisalicylate, Methyl Gentisate, Methyl Salicylate, Metiazinic Acid, Metifenazone, Mofebutazone, Mofezolac, Morazone Hydrochloride, Morniflumate, Morpholine Salicylate, Nabumetone, Naproxen (Naproxen Sodium), Nifenazone, Niflumic Acid, Nimesulide, Oxametacin, Oxaprozin, Oxindanac, Oxyphenbutazone, Parsalmide, Phenybutazone, Phenyramidol Hydrochloride, Picenadol Hydrochloride, Picolamine Salicylate, Piketoprofen, Pirazolac, Piroxicam, Pirprofen, Pranoprofen, Pranosal, Proglumetacin Maleate, Proquazone, Protizinic Acid, Ramifenazone, Salacetamide, Salamidacetic Acid, Salicylamide, Salix, Salol, Salsalate, Sodium Aurothiomalate, Sodium Gentisate, Sodium Salicylate, Sodium Thiosalicylate, Sulindac, Superoxide Dismutase (Orgotein, Pegorgotein, Sudismase), Suprofen, Suxibuzone, Tenidap Sodium, Tenoxicam, Tetrydamine, Thurfyl Salicylate, Tiaprofenic, Tiaramide Hydrochloride, Tinoridine Hydrochloride, Tolfenamic Acid, Tometin Sodium, Triethanolamine Salicylate, Ufenamate, Zaltoprofen, Zidometacin and Zomepirac Sodium.

Further, any of the compounds disclosed herein can be administered in combination or alternation with a second biologically active agent to increase its effectiveness against the target disorder.

In combination therapy, effective dosages of two or more agents are administered together, whereas during alternation therapy an effective dosage of each agent is administered serially. The dosages will depend on absorption, inactivation and excretion rates of the drug as well as other factors known to those of skill in the art. It is to be noted as that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

The efficacy of a drug can be prolonged, augmented, or restored by administering the compound in combination or alternation with a second, and perhaps third, agent that induces a different biological pathway from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the condition.

Any method of alternation can be used that provides treatment to the patient. Nonlimiting examples of alternation patterns include 1-6 weeks of administration of an effective amount of one agent followed by 1-6 weeks of administration of an effective amount of a second agent. The alternation schedule can include periods of no treatment. Combination therapy generally includes the simultaneous administration of an effective ratio of dosages of two or more active agents.

Illustrative examples of specific agents that can be used in combination or alternation with the compounds of the present invention are described below in regard to asthma and arthritis. The agents set out below or others can alternatively be used to treat a host suffering from any of the other disorders listed herein.

The pharmaceutical compositions of the invention can be administered to any animal that can experience the beneficial effects of the compounds of the invention. Foremost among such animals are humans, although the invention is not intended to be so limited.

The pharmaceutical compositions of the present invention can be administered by any means that achieve their intended purpose. For example, administration can be by parenteral, subcutaneous, intravenous, intramuscular, intraperitoneal, transdermal, buccal, or ocular routes. Alternatively, or concurrently, administration can be by the oral route. The dosage administered will be dependent upon the age, health, and weight of the recipient, kind of concurrent treatment, if any, frequency of treatment, and the nature of the effect desired.

In addition to the pharmacologically active compounds, the new pharmaceutical preparations can contain suitable pharmaceutically acceptable carriers comprising excipients and auxiliaries that facilitate processing of the active compounds into preparations that can be used pharmaceutically.

The pharmaceutical preparations of the present invention are manufactured in a manner that is, itself, known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

For compositions of the present invention suitable for administration to a human, the term “excipient” is meant to include, but not be limited by, those excipients described in the Handbook of Pharmaceutical Excipients, American Pharmaceutical Association, 2^{nd} Ed. (1994), which is herein incorporated by reference in its entirety. Suitable excipients are, in particular, fillers such as saccharides, for example, lactose or sucrose, mannitol or sorbitol, cellulose preparations and/or calcium phosphates, for example, tricalcium phosphate or calcium hydrogen phosphate, as well as binders, such as, starch paste, using, for example, maize starch, wheat starch, rice starch, potato starch, gelatin, tragacanth, methyl cellulose, hydroxy-propylmethylcellulose, sodium carboxymethylcellulose, and/or polyvinyl pyrrolidone. If desired, disintegrating agents can be added, such as, the above-mentioned starches and also carboxymethyl-starch, cross-linked polyvinyl pyrrolidone, agar, or alginic acid or a salt thereof, such as, sodium alginate. Auxiliaries are, above all, flow-regulating agents and lubricants, for example, silica, talc, stearic acid or salts thereof, such as, magnesium stearate or calcium stearate, and/or polyethylene glycol. Dragee cores are provided with suitable coatings that, if desired, are resistant to gastric juices. For this purpose, concentrated saccharide solutions can be used, which may optionally contain gum arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as, acetylcellulose phthalate or hydroxypropylmethyl-cellulose phthalate, are used. Dye stuffs or pigments can be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer, such as, glycerol or sorbitol. The push-fit capsules can contain the active compounds in the form of granules that may be mixed with fillers such as lactose, binders such as starches, and/or lubricants such as talc or magnesium stearate and, optionally, stabilizers. In soft capsules, the active compounds are preferably dissolved or suspended in suitable liquids, such as, fatty oils or liquid paraffin. In addition, stabilizers may be added.

Suitable formulations for parenteral administration include aqueous solutions of the active compounds in water-soluble form, for example, water-soluble salts, alkaline solutions and cyclodextrin inclusion complexes. Especially preferred alkaline salts are ammonium salts prepared, for example, with Tris, choline hydroxide, Bis-Tris propane, N-methylglucamine, or arginine. One or more modified or unmodified cyclodextrins can be employed to stabilize and increase the water solubility of compounds of the present invention. Useful cyclodextrins for this purpose are disclosed in U.S. Pat. Nos. 4,727,064, 4,764,604, and 5,024,998.

In addition, suspensions of the active compounds as appropriate oily injection suspensions can be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions can contain substances that increase the viscosity of the suspension, for example, sodium carboxymethyl cellulose, sorbitol, and/or dextran. Optionally, the suspension may also contain stabilizers.

“Pharmaceutically acceptable carriers” for in vivo use are well known in the pharmaceutical art, and are described, for example, in Remington's Pharmaceutical Sciences, Mack Publishing Co. (A. R. Gennaro edit. 1985). The pharmaceutical compositions of the present invention may be formulated with a pharmaceutically acceptable carrier to provide sterile solutions or suspensions for injectable administration. In particular, injectables can be prepared in conventional forms, either as liquid solutions or suspensions, solid forms suitable for solution or suspensions in liquid prior to injection, or as emulsions. Suitable excipients are, for example, water, saline, dextrose, mannitol, lactose, lecithin, albumin, sodium glutamate, cysteine hydrochloride, or the like. In addition, if desired, the injectable pharmaceutical compositions may contain minor amounts of nontoxic auxiliary substances, such as wetting agents, pH buffering agents, and the like. If desired, absorption enhancing preparations (e.g., liposomes) may be utilized.

The present invention also encompasses compositions prepared for storage or administration. These would additionally contain preservatives, stabilizers and dyes. For example, sodium benzoate, sorbic acid and esters of p-hydroxybenzoic acid may be added as preservatives. Id. at 1449. In addition, antioxidants and suspending agents may be used.

The following examples are illustrative, but not limiting, of the method and compositions of the present invention. Other suitable modifications and adaptations of the variety of conditions and parameters normally encountered and obvious to those skilled in the art are within the spirit and scope of the invention.

### EXAMPLE 1

### Tablet Preparation

Tablets containing 25.0, 50.0, and 100.0 mg, respectively, of the following active compounds are prepared as illustrated below:

a. The compound having the structure

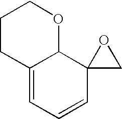

and

b. The compound having the structure

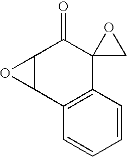

TABLET FOR DOSES CONTAINING FROM
25-100 MG OF THE ACTIVE COMPOUND

| | Amount-mg | | |
|---|---|---|---|
| Active Compound | 25.0 | 50.0 | 100.00 |
| Microcrystalline cellulose | 37.25 | 100.0 | 200.0 |
| Modified food corn starch | 37.25 | 4.25 | 8.5 |
| Magnesium stearate | 0.50 | 0.75 | 1.5 |

All of the active compound, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of active ingredient per tablet.

### EXAMPLE 2

### Intravenous Solution Preparation

An intravenous dosage form of the above-indicated active compounds of Example 1 is prepared as follows:

| | | |
|---|---|---|
| Active Compound | 0.5-10.0 | mg |
| Sodium Citrate | 5-50 | mg |
| Citric Acid | 1-15 | mg |
| Sodium Chloride | 1-8 | mg |
| Water for Injection (USP) | q.s. to 1 | ml |

Utilizing the above quantities, the active compound is dissolved at room temperature in a previously prepared solution of sodium chloride, citric acid, and sodium citrate in Water for Injection (USP, see page 1636 of United States Pharmacopeia/National Formulary for 1995, published by United States Pharmacopeial Convention, Inc., Rockville, Md. (1994).

### EXAMPLE 4

### Activity Assay

Primary human mesangial cells (hMC), purchased from Cambrax Biosciences (Walkersville, Md.), were grown to confluence in mesangial cell basal medium (MSBM; Cambrax Biosciences) containing 5% fetal bovine serum, gentamicin (50 mg/mL) and amphotericin-B (50 mcg/mL) in six-well plates. Prior to adding vehicle, cytokine or test compound, cell monolayers were washed with MSBM to remove serum. All experiments were performed in media consisting of serum-free MSBM plus 0.25% bovine serum albumin (BSA; Sigma Chemical Co., St. Louis).

Recombinant human IL-1° and recombinant human TNF” (R&D Systems, Minneapolis, Minn.) were prepared in phosphate-buffered saline containing 0.1% BSA. The test compounds were dissolved in DMSO or ethanol. The final concentrations of DMSO or ethanol (0.001%) used in these studies did not affect cell viability.

Confluent hMC monolayers in six-well plates were preincubated in a carbon dioxide incubator, in an atmosphere consisting of 95% O₂/5% CO₂, with vehicle or the stipulated concentrations of test compounds for two hours, followed by a 24-hour incubation with vehicle or IL-1β (0.3 ng/mL) or TNFα (5 ng/mL) at 37° C. Stimulus concentrations were taken from the linear phase of the respective concentration-response curves for L-1β- and TNFα-induced MCP-1 (monocyte chemoattractant protein-1) production. Cell-free supernatants were harvested and MCP-1 content was determined using a specific enzyme-linked immunosorbent assay (ELISA). Total cell protein was determined using a kit (DC protein assay; BIO-RAD, Hercules, Calif.), and L-1β- and TNFα-stimulated MCP-1 production was normalized to total cell protein.

Human mesangial cell-free culture supernatants were collected following the incubations, and frozen at −73° C. until use. Immunoreactive MCP-1 protein present in the supernatants was determined using a sandwich ELISA (R&D Systems, Minneapolis, Minn.) according to the manufacturer's protocol, employing a Versa-max microplate reader (Molecular Devices, Sunnyvale, Calif.). Control experiments were carried out to demonstrate that none of the test compounds interfered with the ELISA.

The results were expressed as test compound concentration versus inhibition of MCP-1 production, with data fitted to a sigmoid curve using PRISM software (GraphPad, San Diego, Calif.), and values determined for half-maximal inhibition (IC₅₀) of the maximal obtainable response.

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are fully incorporated by reference herein in their entirety.

What is claimed is:

1. A compound of Formula I: 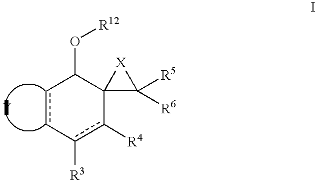
or a solvate, hydrate or pharmaceutically acceptable salt thereof; wherein:
(a) each dotted line indicates the presence of either a single bond or double bond, wherein the valences of a single bond are completed by hydrogens;
(b) X is O, S, or NR¹¹, where R¹¹ is hydrogen or alkyl;
(c) R³, R⁴, R⁵, and R⁶ are independently hydrogen or alkyl;
(d) R¹² is hydrogen, alkyl, carboxylalkyl, or R¹² is absent; and
(e) Y, together with the two ring carbon atoms to which it is attached, represents a 5- to 7-membered monocyclic carbon ring which is saturated, partially saturated, or aromatic.

2. A compound of claim 1, wherein X is O.

3. A compound of claim 1, wherein Y is phenyl or cyclohexyl.

4. A compound of claim 1, wherein R³, R⁴, R⁵, and R⁶ are independently hydrogen.

5. A compound of claim 1, wherein Y is phenyl and R³, R⁴, R⁵, and R⁶ are each hydrogen.

6. A compound of claim 1 that is one of: 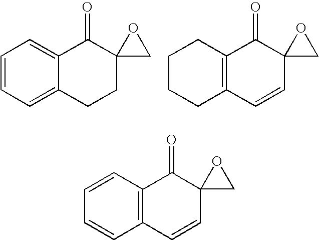
or a solvate, hydrate or pharmaceutically acceptable salt thereof.

7. A compound of Formula II: 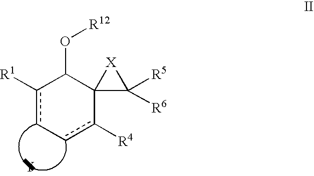
or a solvate, hydrate or pharmaceutically acceptable salt thereof; wherein:
(a) the dotted line indicates the presence of either a single bond or double bond, wherein the valences of a single bond are completed by hydrogens;
(b) X is O, S, or NR¹¹, where R¹¹ is hydrogen or alkyl;
(c) R¹, R⁴, R⁵, and R⁶ are independently hydrogen or alkyl;
(d) R¹² is hydrogen, alkyl, carboxylalkyl, or R¹² is absent; and
(e) Y, together with the two ring carbon atoms to which it is attached, represents a 5- to 7-membered monocyclic carbon ring which can be saturated, partially saturated, or aromatic.

8. The compound of claim 7, wherein X is O.

9. The compound of claim 7, wherein R¹, R⁴, R⁵, and R⁶ are independently hydrogen.

10. The compound of claim 7, wherein Y is phenyl.

11. The compound of claim 7 that is 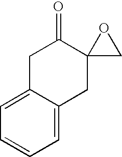
or a solvate, hydrate or pharmaceutically acceptable salt thereof.

12. A compound of Formula III: 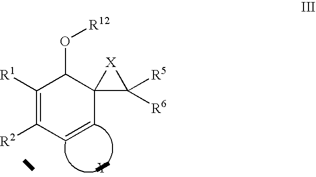
or a solvate, hydrate or pharmaceutically acceptable salt thereof; wherein:
(a) X is O, S, or NR¹¹, where R¹¹ is hydrogen or alkyl;
(b) R¹, R², R⁵, and R⁶ are independently hydrogen or alkyl;
(c) R¹² is hydrogen, alkyl, carboxylalkyl, or R¹² is absent; and
(d) Y, together with the two ring carbon atoms to which it is attached, represents a 5- to 7-membered monocyclic carbon ring which can be saturated, partially saturated, or aromatic.

13. The compound of claim 12, wherein X is O.

14. The compound of claim 12, wherein R¹, R², R⁵, and R⁶ are each hydrogen.

15. The compound of claim 12, wherein Y is phenyl.

16. The compound of claim 12 that is 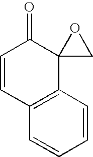
or a solvate, hydrate or pharmaceutically acceptable salt thereof.

17. A compound of Formula IV: 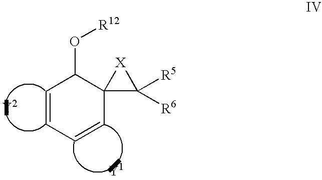
or a solvate, hydrate or pharmaceutically acceptable salt thereof; wherein:
(a) X is O, S, or NR¹¹, where R¹¹ is hydrogen or alkyl;
(b) R⁵ and R⁶ are independently hydrogen or alkyl;
(c) R¹² is hydrogen, alkyl, carboxylalkyl, or R¹² is absent; and
(d) Y¹ and Y² each represent, together with the two ring carbon atoms to which each is attached, a 5- to 7-membered monocyclic carbon ring which can be saturated, partially saturated, or aromatic.

18. The compound of claim 17, wherein X is O.

19. The compound of claim 17, wherein R⁵ and R⁶ are each hydrogen.

20. The compound of claim 17, wherein Y¹ and Y² are each C₆ aryl.

21. The compound of claim 17 that is 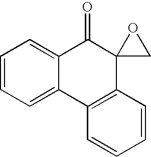
or a solvate, hydrate or pharmaceutically acceptable salt thereof.

22. A compound of Formula V: 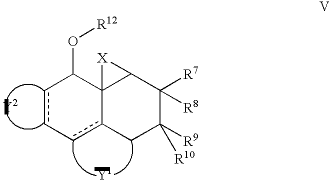
or a solvate, hydrate or pharmaceutically acceptable salt thereof; wherein:
(a) the dotted line indicates the presence of either a single bond or double bond, wherein the valences of a single bond are completed by hydrogens;
(b) X is O, S, or NR¹¹, where R¹¹ is hydrogen or alkyl;
(c) R⁵, R⁷, R⁸, R⁹, and R¹⁰ are independently hydrogen or alkyl;
(d) R¹² is hydrogen, alkyl, carboxylalkyl, or R¹² is absent;
(e) Y¹ represents, together with the two ring carbon atoms to which it is attached, a 5- to 7-membered monocyclic carbon ring which can be saturated, partially saturated, or aromatic; and
(f) Y² represents, together with the three ring carbon atoms to which it is attached, a 5- to 7-membered monocyclic carbon ring which can be saturated, partially saturated, or aromatic.

23. The compound of claim 22, wherein X is O.

24. The compound of claim 22, wherein R⁵, R⁷, R⁸, R⁹, and R¹⁰ are each hydrogen.

25. The compound of claim 22, wherein Y¹ and Y² are each phenyl.

26. The compound of claim 22 that is 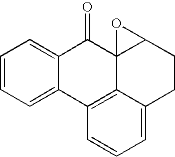
or a solvate, hydrate or pharmaceutically acceptable salt thereof.

27. A compound of Formula VI: 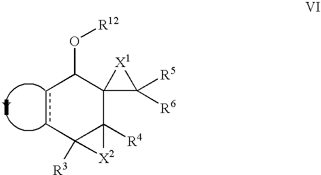
or a solvate, hydrate or pharmaceutically acceptable salt thereof; wherein:
(a) the dotted line indicates the presence of either a single bond or double bond, wherein the valences of a single bond are completed by hydrogens;
(b) X¹ and X² are independently O, S, or NR¹¹, where R¹¹ is hydrogen or alkyl;
(c) R³, R⁴, R¹ and R⁶ are independently hydrogen or alkyl;
(d) R¹² is hydrogen, alkyl, carboxylalkyl, or R¹² is absent; and
(e) Y, together with the two ring carbon atoms to which it is attached, represents a 5- to 7-membered monocyclic carbon ring which can be saturated, partially saturated, or aromatic.

28. The compound of claim 27, wherein .X¹ and X² are each O.

29. The compound of claim 27, wherein R³, R⁴, R¹ and R⁶ are independently hydrogen.

30. The compound of claim 27, wherein Y is phenyl.

31. The compound of claim 27 that is one of: 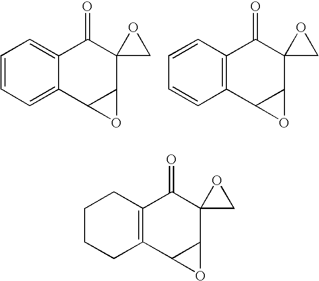
or a solvate, hydrate or pharmaceutically acceptable salt thereof.

32. A compound of Formula VII: 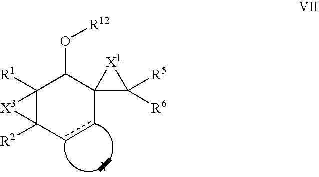
or a solvate, hydrate or pharmaceutically acceptable salt thereof; wherein:
(a) X¹ and X³ are independently O, S, or NR¹¹, where R¹¹ is hydrogen or alkyl;
(b) R¹, R², R⁵, and R⁶ are independently hydrogen or alkyl;
(c) R¹² is hydrogen, alkyl, carboxylalkyl, or R¹² is absent; and
(d) Y, together with the two ring carbon atoms to which it is attached, represents a 5- to 7-membered monocyclic carbon ring which can be saturated, partially saturated, or aromatic.

33. The compound of claim 32, wherein X¹ and X³ are independently O.

34. The compound of claim 32, wherein R¹, R², R⁵, and R⁶ are independently hydrogen.

35. The compound of claim 32, wherein Y is phenyl.

36. The compound of claim 32 that is 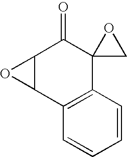
or a solvate, hydrate or pharmaceutically acceptable salt thereof.

37. A compound of Formula VIII: 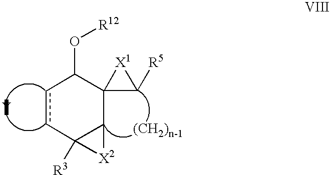
or a solvate, hydrate or pharmaceutically acceptable salt thereof; wherein:
(a) the dotted line indicates the presence of either a single bond or double bond, wherein the valences of a single bond are completed by hydrogens;
(b) X¹ and X² are independently O, S, or NR¹¹, where R¹¹ is hydrogen or alkyl;
(c) R³ and R⁵ are independently hydrogen or alkyl;
(d) R¹² is hydrogen, alkyl, carboxylalkyl, or R¹² is absent;
(e) Y, together with the two ring carbon atoms to which it is attached, represents a 5- to 7-membered monocyclic carbon ring which can be saturated, partially saturated, or aromatic; and
(f) n is from 3 to 5.

38. The compound of claim 37, wherein X¹ and X² are each O.

39. The compound of claim 37, wherein Y is phenyl.

40. The compound of claim 37, wherein R³ and R⁵ are each hydrogen and n is 4.

41. The compound of claim 37 that is 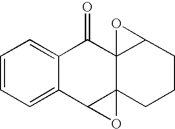
or a solvate, hydrate or pharmaceutically acceptable salt thereof.

42. A compound of Formula IX: 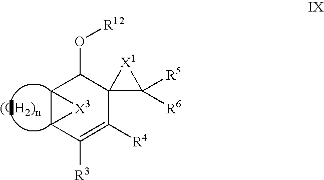
or a solvate, hydrate or pharmaceutically acceptable salt thereof; wherein:
(a) X¹ and X³ are independently O, S, or NR¹¹, where R¹¹ is hydrogen or alkyl;
(b) R³, R⁴, R⁵, and R⁶ are independently hydrogen or alkyl;
(c) R¹² is hydrogen, alkyl, carboxylalkyl, or R¹² is absent; and
(d) n is from 3 to 5.

43. The compound of claim 42, wherein X¹ and X³ are each O.

44. The compound of claim 42, wherein n is 4.

45. The compound of claim 42 that is 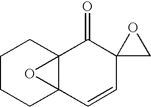
or a solvate, hydrate or pharmaceutically acceptable salt thereof.

46. A compound of Formula X: 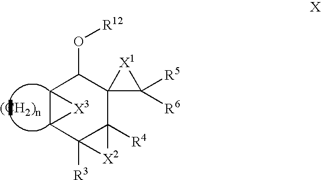
or a solvate, hydrate or pharmaceutically acceptable salt thereof; wherein:
(a) X¹, X², and X³ are independently O, S, or NR¹¹, where R¹¹ is hydrogen or alkyl;
(b) R³, R⁴, R⁵, and R⁶ are independently hydrogen or alkyl;
(c) R¹² is hydrogen, alkyl, carboxylalkyl, or R² is absent; and
(d) n is from 3 to 5.

47. The compound of claim 46, wherein X¹, X², and X³ are each O.

48. The compound of claim 46, wherein n is 4.

49. The compound of claim 46 that is 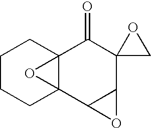
or a solvate, hydrate or pharmaceutically acceptable salt thereof.

50. A compound of Formula XI: 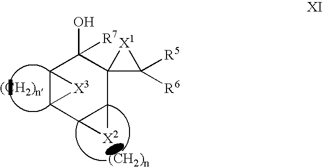
or a solvate, hydrate or pharmaceutically acceptable salt thereof; wherein:
(a) X¹, X², and X³ are independently O, S, or NR¹¹, where R¹¹ is hydrogen or alkyl;
(b) R⁵, R⁶, and R⁷ are independently hydrogen or alkyl; and
(c) n and n′ are each from 3 to 5.

51. The compound of claim 50, wherein X¹, X², and X³ are each O.

52. The compound of claim 50, wherein R⁵, R⁶, and R⁷ are each hydrogen.

53. The compound of claim 50, wherein n and n′ are each 4.

54. The compound of claim 50 that is 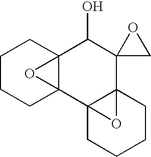
or a solvate, hydrate or pharmaceutically acceptable salt thereof.

55. A compound of Formula XII: 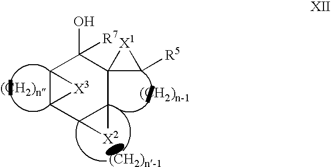
or a solvate, hydrate or pharmaceutically acceptable salt thereof; wherein:
(a) X¹, X², and X³ are independently O, S, or NR¹¹, where R¹¹ is hydrogen or alkyl;
(b) R⁵ and R⁷ are independently hydrogen or alkyl; and
(c) n, n′ and n″ are each from 3 to 5.

56. The compound of claim 55, wherein X¹, X², and X³ are each O.

57. The compound of claim 55, wherein R⁵ and R⁷ are each hydrogen.

58. The compound of claim 55, wherein n, n′, and n″ are each 4.

59. The compound of claim 55 that is 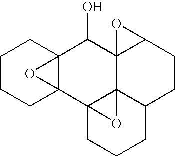
or a solvate, hydrate or pharmaceutically acceptable salt thereof.

60. A compound of Formula XIII: 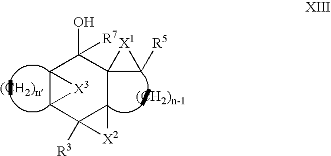
or a solvate, hydrate or pharmaceutically acceptable salt thereof; wherein:
(a) X¹, X², and X³ are independently O, S, or NR¹¹, where R¹¹ is hydrogen or alkyl;
(b) R³, R⁵, and R⁷ are independently hydrogen or alkyl; and
(c) n and n′ are each from 3 to 5.

61. The compound of claim 60, wherein X¹, X², and X³ are each O.

62. The compound of claim 60, wherein R³, R⁵, and R⁷ are each hydrogen.

63. The compound of claim 60, wherein n and n′ are each 4.

64. The compound of claim 60 that is 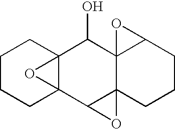
or a solvate, hydrate or pharmaceutically acceptable salt thereof.

65. A compound of Formula XIV: 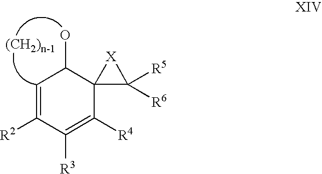
or a solvate, hydrate or pharmaceutically acceptable salt thereof; wherein:
(a) X is O, S, or NR¹¹, where R¹¹ is hydrogen or alkyl;
(b) R², R³, R⁴, R⁵, and R⁶ are independently hydrogen or alkyl; and
(c) n is from 3 to 5.

66. The compound of claim 65, wherein X is O.

67. The compound of claim 65, wherein R², R³, R⁴, R⁵, and R⁶ are each hydrogen.

68. The compound of claim 65, wherein n is 4.

69. The compound of claim 65 that is 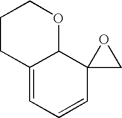
or a solvate, hydrate or pharmaceutically acceptable salt thereof.

70. A compound of Formula XV: 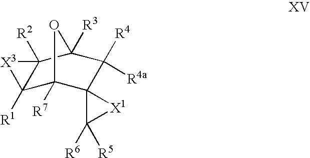
or a solvate, hydrate or pharmaceutically acceptable salt thereof, wherein:
(a) X¹ and X³ are independently O, S, or NR¹¹, where R¹¹ is hydrogen or alkyl; and
(b) R¹, R², R³, R⁴, R^{4a}, R⁵, R⁶, and R⁷ are independently hydrogen or alkyl.

71. The compound of claim 70, wherein X¹ and X³ are each O.

72. The compound of claim 70, wherein R¹, R², R³, R⁴, R⁴, R⁵, R⁶, and R⁷ are each hydrogen.

73. The compound of claim 70 that is 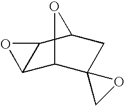
or a solvate, hydrate or pharmaceutically acceptable salt thereof.

74. A compound of Formula XVI: 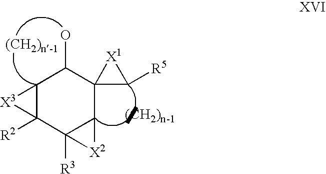
or a solvate, hydrate or pharmaceutically acceptable salt thereof; wherein:
(a) X¹, X², and X³ are independently O, S, or NR¹¹, where R¹¹ is hydrogen or alkyl;
(b) R², R³, and R⁵ are independently hydrogen or alkyl; and
(c) n and n′ are each from 3 to 5.

75. The compound of claim 74, wherein X¹, X², and X³ are each O.

76. The compound of claim 74, wherein R², R³, and R⁵ are each hydrogen.

77. The compound of claim 74, wherein n and n′ are each 4.

78. The compound of claim 74 that is 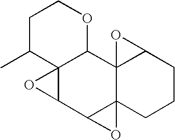
or a solvate, hydrate or pharmaceutically acceptable salt thereof.

79. A compound of Formula XVII: 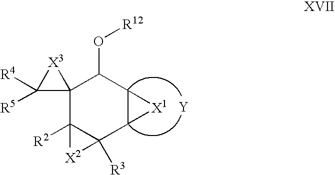
or a solvate, hydrate or pharmaceutically acceptable salt thereof; wherein:
(a) X¹, X², and X³ are independently O, S, or NH;
(b) R², R³, R⁴, and R⁵ are independently hydrogen or alkyl;
(c) R¹² is hydrogen, alkyl, carboxylalkyl, or R¹² is absent; and
(d) Y, together with the two ring carbon atoms to which it is attached, represents a 5- to 7-membered monocyclic carbon ring which is saturated, or partially saturated.

80. A compound of claim 79, wherein X¹, X², and X³ are each O.

81. A compound of claim 79, wherein R², R³, R⁴, and R⁵ are each hydrogen.

82. The compound of claim 79, wherein Y is cyclohexyl.

83. The compound of claim 79 that is:

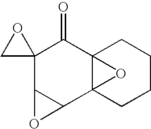

84. A pharmaceutical composition comprising a compound of any of claims 1-83 and a pharmaceutically acceptable carrier.

85. A method for the treatment of prophylaxis of an inflammatory disorder in a mammal in need thereof, comprising administering to said mammal an effective amount of a composition of claim 84.

86. A method for the treatment or prophylaxis of an autoimmune disorder in a mammal in need thereof, comprising administering to said mammal an effective amount of a composition of claim 84.
